(19) United States
(12) Patent Application Publication
(10) Pub. No.: US 2026/0357228 A1
(43) Pub. Date: Nov. 12, 2026

(54) CRISPR/NCAS9-EDITED CD34+ CELLS AND METHODS FOR PHENOTYPIC RESCUE OF MPS-IVA FIBROBLASTS

(71) Applicant: The Nemours Foundation, Jacksonville, FL (US)

(72) Inventors: Shunji TOMATSU, Wilmington, DE (US); Andres Felipe Leal BOHORQUEZ, Bogota (CO)

(21) Appl. No.: 19/662,737

(22) Filed: Apr. 29, 2026

Related U.S. Application Data
(60) Provisional application No. 63/797,251, filed on Apr. 30, 2025.

(51) Int. Cl. C12N 9/16; C12N 5/0775

(52) U.S. Cl. CPC C12N 9/16; C12N 5/0663; C12Y 301/06004; C12N 2502/1323; C12N 2510/00

(57) ABSTRACT

Provided and described herein are compositions and methods for the sustained delivery of GALNS enzyme via genetically modified CD34+ stem cells that express the GALNS enzyme. The modified CD34+ cells provided and described herein are generally useful for increasing or rescuing GALNS activity, increasing glycosaminoglycans (GAG) clearance, reducing mitochondrial and/or metabolic stress, improving cell survival, and/or reducing inflammation associated with a GALNS deficiency. Moreover, such advantageous properties can be achieved while maintaining the stemness of the CD34+ stem cells.

### CROSS-REFERENCE TO RELATED APPLICATIONS

This application claims the benefit of priority to U.S. Provisional Patent Application No. 63/797,251, filed on Apr. 30, 2025, the entire contents of which are hereby incorporated by reference in their entirety.

### STATEMENT OF GOVERNMENTAL INTEREST

The invention described herein was supported by the United States National Institutes of Health Grant No. 1-R01-HD102545. The United States government has certain rights in the invention.

### INCORPORATION BY REFERENCE OF SEQUENCE LISTING

The instant application contains a Sequence Listing which has been submitted electronically in XML file format and is hereby incorporated by reference in its entirety. The XML copy, created on Apr. 27, 2026, is named 045009-0073-US-00-000064_SL.xml and is 4,889 bytes in size.

### BACKGROUND

Mucopolysaccharidosis (MPS) IVA (OMIM: 253000) is a genetic lysosomal storage disease (LSD) caused by a deficiency in the N-acetylgalactosamine-6-sulfatase (GALNS) enzyme that leads to the lysosomal accumulation of the glycosaminoglycans (GAGs), keratan sulfate (KS) and chondroitin 6-sulfate (C6S). The accumulation of KS and C6S disrupts the function of chondrocytes in the growth plates, impairs cartilage matrix development, and inhibits bone matrix mineralization. The resultant incomplete or defective endochondral ossification and successive imbalance of growth in MPS IVA patients cause unique skeletal symptoms such as prominent forehead, abnormal face with a large mandible, disproportionate short-trunk dwarfism with short neck, cervical spine instability with odontoid hypoplasia, cervical spinal cord compression, pectus carinatum, tracheal deviation and obstruction, re-strictive lung, flaring of the rib cage, kyphoscoliosis, platyspondyly, hip dysplasia with coxa valga, genu valgum, hypermobile joints, waddling gait, and pes planus. The degree of imbalance of growth in bone and other organs and tissues largely contributes to unique skeletal dysplasia and clinical severity, including narrowing trachea.

### SUMMARY

Currently, enzyme replacement therapy (ERT) is the sole approved treatment for Mucopolysaccharidosis (MPS) IVA. However, ERT presents significant clinical limitations, as it is hindered by rapid hepatic and splenic clearance, the induction of neutralizing antibodies, and poor penetration into skeletal tissues. While allogeneic hematopoietic stem cell (HSC) transplantation has also been contemplated as an alternative therapeutic option, it is severely restricted by the requirement for HLA-matched donors and presents the high risk of graft-versus-host disease (GVHD).

Accordingly, provided and described herein are compositions and methods for the sustained delivery of GALNS enzyme via genetically modified CD34+ stem cells that express the GALNS enzyme. The modified CD34+ cells provided and described herein are generally useful for increasing or rescuing GALNS activity, increasing glycosaminoglycans (GAG) clearance, reducing mitochondrial and/or metabolic stress, improving cell survival, and/or reducing inflammation associated with a GALNS deficiency. Moreover, such advantageous properties can be achieved while maintaining the stemness of the CD34+ stem cells. Such modified CD34+ cells and methods, of use, and methods of manufacture provide a robust platform (e.g., an ex vivo autologous cell therapy platform) for increasing GALNS enzyme activity and/or for the sustained rescue of GALNS-deficient tissues (e.g., in MPS IVA).

In some embodiments, provided and described herein are modified CD34 positive (CD34+) cells comprising an exogenous polynucleotide, wherein: (i) the exogenous polynucleotide comprises a protein coding sequence that encodes a N-acetylgalactosamine-6-sulfatase (GALNS) enzyme; and (ii) the exogenous polynucleotide is integrated into the genome of the CD34+ cells.

In certain embodiments, the GALNS enzyme comprises an amino acid sequence having at least 90% sequence identity to SEQ ID NO: 1. In certain embodiments, the GALNS enzyme hydrolyzes a sulfate group from a 4-methylumbelliferyl-β-D-galactopyranoside-6-sulfate (4MU-Gal-6S) substrate (e.g., in a fluorometric assay). In certain embodiments, the exogenous polynucleotide further comprises a promoter sequence, wherein the promoter sequence initiates transcription of the protein coding sequence encoding the GALNS enzyme.

In certain embodiments, the CD34+ is a CD34+ stem cell. In certain embodiments, the CD34+ stem cell is a bone marrow CD34+ stem cell.

In certain embodiments, the exogenous polynucleotide is integrated into a genomic safe harbor site within the genome. In certain embodiments, the genomic safe harbor site is located within an intragenic site and open chromatin.

In certain embodiments, the modified CD34+ cell secretes the GALNS enzyme. In certain embodiments, co-culturing of the modified CD34+ cell with co-culture MPS IVA fibroblasts results in an increase in the GALNS activity in the MPS IVA fibroblasts. In certain embodiments, the GALNS activity is extracellular GALNS activity, intracellular GALNS activity, or both extracellular GALNS activity and intracellular GALNS activity. In certain embodiments, the modified CD34+ cell is capable of forming colony-forming units (CFUs), wherein the CFUs comprise one or more of CFU-GEMM, CFU-GM, BFU-E, CFU-G, and CFU-M.

In some embodiments, provided and described herein are methods of increasing GALNS activity of GALNS-deficient cells, the method comprising: co-localizing the modified CD34+ cells with the GALNS-deficient cells. In certain embodiments, the method results in increased GALNS activity. In certain embodiments, the GALNS activity is extracellular GALNS activity, intracellular GALNS activity, or both extracellular GALNS activity and intracellular GALNS activity.

In some embodiments, provided and described herein are methods of reducing glycosaminoglycan (GAG) accumulation in GALNS-deficient cells, the method comprising: the method comprising: co-localizing the modified CD34+ cells with the GALNS-deficient cells. In certain embodiments, the method results in a reduction in GAG accumulation in the GALNS-deficient cells. In certain embodiments, the GAG comprises keratan sulfate (KS). In certain embodiments, the KS is mono-KS.

In some embodiments, provided and described herein are methods of reducing metabolic stress in GALNS-deficient cells, the method comprising: co-localizing the modified CD34+ cells with the GALNS-deficient cells. In certain embodiments, the method results in a reduction of metabolic stress within the GALNS-deficient cells. In certain embodiments, the method results in a reduction of mitochondrial reactive oxygen species within the GALNS-deficient cells. In certain embodiments, the method results in a reduction of mitochondrial mass of the GALNS-deficient cells. In certain embodiments, the method results in a reduction in cell death of the GALNS-deficient cells.

In some embodiments, provided and described herein are methods of reducing pro-inflammatory proteins within GALNS-deficient cells, the method comprising: co-localizing the modified CD34+ cells with the GALNS-deficient cells. In certain embodiments, the method results in a reduction of pro-inflammatory biomarkers within the GALNS-deficient cells. In certain embodiments, the pro-inflammatory biomarkers comprise Interleukin-1 beta (IL1b) or interleukin-6 beta (IL6b).

In some embodiments, provided and described herein are of making a modified CD34+ cell, the method comprising: cutting the genome of a CD34+ cell using a targetable gene editing system to generate a cut site; and providing an exogenous polynucleotide to the CD34+ cell, wherein the exogenous polynucleotide comprises a protein coding sequence that encodes a N-acetylgalactosamine-6-sulfatase (GALNS) enzyme and is inserted into the genome at the cut site. In certain embodiments, the targetable gene editing system comprises a CRISPR-Cas gene editing system. In certain embodiments, the CRISPR-Cas gene editing system comprises: (i) a first CRISPR-Cas complex comprising a first CRISPR-Cas nuclease and a first guide RNA (gRNA) that targets the first nuclease to a first site within the cut site; (ii) a second CRISPR-Cas complex comprising a second CRISPR-Cas nuclease and a second guide RNA (gRNA) that targets the second nuclease to a second site within the cut site. In certain embodiments, the cut site is within a genomic safe harbor site of the genome. In certain embodiments, the genomic safe harbor site is located within an intragenic site and open chromatin. In certain embodiments, providing an exogenous polynucleotide to the CD34+ cell comprises transfecting the CD34+ cell with the exogenous polynucleotide. In certain embodiments, the method is an ex vivo method.

In some embodiments, provided and described herein are methods of increasing an amount of GALNS enzyme in an individual having a GALNS deficiency, the method comprising: administering the modified CD34+ cells to the individual. In certain embodiments, the method results in an increased amount of GALNS in the individual. In certain embodiments, the GALNS deficiency is MPS IVA.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein) of which:

FIGS. 1A, 1B, 1C, and 1D show generation of modified CD34+ cells having an integrated exogenous polynucleotide encoding a GALNS enzyme, along with GLANS activity data. FIG. 1A shows a schematic representation of CRISPR/nCas9 system strategy and two sgRNAs targeting the AAVS1 locus and PAM sequence (top), AAV6-donor template construct, two homologous arms flanked an expression cassette containing an hPGK promoter, GALNS cDNA, and the bovine growth hormone (bGH) poly(A) signal sequence (bottom). FIG. 1B shows a histogram from CD34+ cells positive for GFP (left). The percentage of GFP-positive CD34+ cells with AAV6-donor-EGFP and with or without the RNP-based CRISPR/nCas9 (right) (n=6). FIGS. 1C and 1D show extracellular and intracellular GALNS activity in CD34+ cells with the RNP-based CRISPR/nCas9 or AAV6-donor (n=5). Results are expressed as fold-change to untransfected CD34+ cells. The dotted line represents the untransfected CD34+ cell levels detected. *p<0.05, #p<0.001.

FIGS. 2A, 2B, 2C, 2D, and 2E show the evaluation of stemness and differentiation potential the modified CD34+ cells. FIG. 2A shows the percentage of cells positive for CD34, CD38, and CD45 surface markers (n=6). FIG. 2B shows the differentiation potential as determined by the expression levels of differentiation markers CD15, CD14, and CD235a (n=12), including the total number of colonies formed per plate (left), as well as the number of colony types representing progenitor and mature multilineage colonies (right). FIG. 2C shows phase-contrast microscopy images of representative burst-forming unit-erythroid (BFU-E), colony-forming unit-granulocyte (CFU-G), and colony-forming unit-monocyte (CFU-M) in CD34+ cells transfected with or without CRISPR/nCas9. The scale bar represents 50 μm. FIG. 2D shows the proliferation index of the cells (n=5). FIG. 2E presents the cell cycle distribution in the edited CD34+ cells (n=5). “Mock” refers to unedited CD34+ cells. “ns” indicates no significant differences.

FIGS. 3A, 3B, 3C, 3D, 3E, and 3F show GALNS enzyme activity and lysosomal mass evaluation in MPS IVA fibroblasts co-cultured with modified CD34+ cells. FIG. 3A shows a schematic representation of the experimental process, including electroporation of CD34+ cells with a ribonucleoprotein (RNP) complex comprising CRISPR/nCas9 and two sgRNAs targeting the AAVS1 locus at a 1:2.5 molar ratio, transduction of the AAV6 donor template at an MOI of 15,000, and subsequent co-culture of MPS IVA fibroblasts with CRISPR/nCas9-edited CD34+ cells for up to a month. FIGS. 3B and 3C show extracellular and intracellular GALNS activity in MPS IVA fibroblasts (n=6). “Mock” refers to unedited CD34+ cells. FIG. 3D shows mono-sulfated keratan sulfate (KS) levels (n=3). FIG. 3E shows representative histograms (left) and mean fluorescence intensity (MFI) (right) of lysosomal mass detected by flow cytometry (n=4). FIG. 3F shows epifluorescence microscopy images for lysosomal mass and nucleus, with a scale bar representing 20 μm. Results are expressed as fold-change to wild-type (WT) fibroblasts. The dotted line represents WT fibroblast levels. *p<0.05, **p<0.005.

FIGS. 4A, 4B, 4C, and 4D show pro-metabolic (-oxidant) profile assessment in MPS IVA fibroblasts co-cultured with modified CD34+ cells. FIG. 4A shows detection of reactive oxygen species (ROS), including a representative histogram (left) and mean fluorescence intensity (MFI) of H2DCFDA fluorescence probe by flow cytometry (right) (n=6). FIG. 4B shows detection of mitochondrial-related oxidative stress (mtROS), including a representative histogram (left) and MFI (right) of MitoSOX red fluorescence by flow cytometry (n=6). FIG. 4C shows detection of mitochondrial mass, with a representative histogram (left) and MFI (right) of Nonyl Acridine Orange (NAO) by flow cytometry (n=6). FIG. 4D shows apoptosis assays, including the gating strategy (left) and mean percentages of apoptotic cells (right) (n=6). Results are expressed as fold-change to wild-type (WT) fibroblasts. The dotted line represents WT fibroblast levels. “PC” indicates positive control. *p<0.05, **p<0.005, ##p<0.0001.

FIGS. 5A and 5B show pro-inflammatory profile evaluation in MPS IVA fibroblasts co-cultured with modified cells CD34+ cells. FIG. 5A shows levels of Interleukin-1 beta (IL-1β) (n=7). FIG. 5B shows levels of Interleukin-6 (IL-6) (n=8). Results are expressed as fold-change to wild-type (WT) fibroblasts. The dotted line represents WT fibroblast levels. *p<0.05, ##p<0.0001.

FIGS. 6A and 6B show on-target efficiency of the CRISPR/nCas9 RNP complex. FIG. 6A shows an agarose gel depicting the on-target efficiency of sgRNAs targeting the AAVS1 locus in HEK-293 cells, as detected by T7-base endonuclease assay. “PC” indicates positive control. FIG. 6B shows transduction efficiency of AAV6-Donor-EGFP at different multiplicities of infection (MOIs), including a representative histogram of GFP-positive HEK-293 cells (left) and the mean percentage of GFP-positive HEK-293 cells.

FIG. 7 shows the percentage of live cells post-editing in CD34+ cells transfected with RNP-based CRISPR/nCas9 and AAV6-donor alone. Cell viability was evaluated by PI in flow cytometry. *p<0.05, **p<0.005.

FIGS. 8A and 8B show a representative gating strategy of CD34+ cells for the analysis of CD34+, CD38+, and CD45+ marker in CD34+ cells transfected without or with RNP-based CRISPR/nCas9 system.

### DETAILED DESCRIPTION

Despite breakthrough progress in MPS IVA therapies, less than desired long-term efficacy remains, as current treatments are hindered by rapid systemic clearance, immunogenicity, and poor bone distribution. For example, less than desired therapeutic profiles are realized due to the rapid clearance and short half-life of infused enzymes. Accordingly, provided and described herein are compositions and methods for the sustained delivery of GALNS enzyme via CD34+ stem cells genetically modified to express the GALNS enzyme. The modified CD34+ cells provided and described herein are generally useful for increasing or rescuing GALNS activity, increasing glycosaminoglycans (GAG) clearance, reducing mitochondrial and/or oxidative stress, improving cell survival, and/or reducing inflammation associated with a GALNS deficiency, when co-localized with GALNS-deficient cells. Such advantageous properties can be achieved while maintaining the stemness of the CD34+ stem cells. The modified CD34+ cells and methods, of use, and methods of manufacture generally provide a robust platform (e.g., ex vivo therapeutic platform) for increasing GALNS enzyme and/or for the sustained rescue of GALNS-deficient tissues (e.g., in MPS IVA).

### Modified CD34+ Cells

As described herein and exemplified in the Examples, genetically modified CD34+ cells that express a GALNS enzyme (modified CD34+ cells) can be engineered to generally achieve a stable source providing a functional GALNS enzyme to mediate the cross-correction of GALNS-deficient cells. Notably, the modified CD34+ cells also preserve, in certain instances, the fundamental stemness, cellular proliferation, and multilineage capacities of hematopoietic progenitors. By providing a stable, continuous supply of GALNS enzyme, the modified cells can provide for the biochemical rescue of GALNS-deficient cells. For example, such biochemical rescue includes, but is not limited to, increasing GALNS amount, increasing GALNS activity (e.g., clearing of accumulated substrates), normalizing lysosomal and mitochondrial pathologies (e.g., oxidative stress), and/or reducing both apoptotic and inflammatory profiles in GALNS-deficient cells.

Expression of the GALNS enzyme in the modified CD34+ cells is generally achieved by integrating an exogenous polynucleotide having a protein coding sequence that encodes the GALNS enzyme into the genome of a CD34+ cells. A GALNS enzyme (also referred to as N-acetylgalactosamine-6-sulfatase) generally refers to and includes the enzyme capable of hydrolyzing the 6-sulfate groups of the N-acetyl-D-galactosamine 6-sulfate units of chondroitin sulfate and/or of the D-galactose 6-sulfate units of keratan sulfate. The GALNS enzyme protein sequence generally refers to and includes a protein sequence having at least about 85% sequence identity to the GALNS enzyme referenced in UniProt ID P34059 or at least about 85% sequence identity to SEQ ID NO: 1. Activity of a GALNS enzyme can be determined by, in certain instances, using a standard fluorometric assay that quantifies the enzyme's ability to cleave a specific synthetic substrate, such as 4-methylumbelliferyl-β-D-galactopyranoside-6-sulfate (4-MU-Gal-6S). The enzymatic cleavage releases a fluorescent product (4-MU) and the signal intensity is measured via a fluorometer, providing a readout that is directly proportional to the amount of functional GALNS present in the cells or secreted into the environment. Such an assay is provided in the Examples.

Non-limiting examples of a GALNS enzyme include a GALNS enzyme comprising an amino acid sequence having at least 90%, at least 95%, at least 97%, at least 98%, at least 99%, or 100% sequence identity to SEQ ID NO: 1. Exemplary nucleic acid sequences that encode a GALNS enzyme (e.g., having at least 90%, at least 95%, at least 97%, at least 98%, or at least 99% sequence identity to SEQ ID NO: 1) include polynucleotides (i) having at least 70%, at least 80%, at least 90%, at least 95%, at least 98%, at least 99%, or 100% sequence identity to SEQ ID NO: 2.

A CD34+ cell generally refers to and includes a stem cell that expresses the CD34 antigen. In certain instances, the CD34+ cell possesses a primitive immunophenotype and/or lacks mature lineage markers (Lin−). Functionally, CD34+ cells generally exhibit fundamental stemness, characterized by the capacity for self-renewal and the multipotent capacity to differentiate into diverse multilineage hematopoietic colonies (e.g., myeloid, erythroid, and lymphoid lineages). Stemness can be measured or demonstrated by colony forming unit assays and/or flow cytometry characterization. A Colony-Forming Unit (CFU) assay demonstrates multipotency by culturing the modified CD34+ cells in a semi-solid medium to confirm they can still differentiate into diverse, multilineage hematopoietic colonies. A count of these colonies (e.g., erythroid and myeloid), specifically CFU-E, CFU-GM, and CFU-GEMM, demonstrate the capacity to into diverse multilineage hematopoietic colonies. Flow cytometry can be used to analyze the cell surface markers, confirming the modified cells maintain high expression of CD34 and/or confirming the ability to differentiate into different hematopoietic linages via biomarker characterization. Such assays are provided in the Examples.

Examples of CD34+ cells include, but are not limited to, CD34+ stem cells. Non-limiting examples of CD34+ stem cells include CD34+ hematopoietic stem cells. Examples of CD34+ hematopoietic stem cells include bone marrow-derived CD34+ stem cells.

A GALNS-deficient cell generally refers to and includes any cell (e.g., human cell) characterized by a complete absence, or a reduction (i.e., a pathogenic reduction), of a functional N-acetylgalactosamine-6-sulfatase (GALNS) enzyme and/or GALNS enzymatic activity. This deficiency is typically the result of one or more mutations (e.g., missense, nonsense, insertion, deletion, or splice-site mutations) in the GALNS gene located on chromosome 16q24.3. A cell is considered GALNS-deficient if its measurable GALNS specific activity falls substantially below a wild-type, healthy cell (e.g., 25% less, 50% less, or 100% less than wild-type).

Mucopolysaccharidosis Type IVA or MPS IVA generally comprises any pathological condition, disease state, or syndrome fundamentally caused by a deleterious mutation, ablation, or functional deficiency in the GALNS gene (encoding the enzyme N-acetylgalactosamine-6-sulfatase). The MPS IVA disease state can be defined at the cellular and biochemical level by the inability of lysosomes to effectively catabolize keratan sulfate and chondroitin-6-sulfate, leading to toxic intracellular accumulation, particularly within chondrocytes and the extracellular matrix of cartilage and bone. An MPS IVA designation can encompass any genotypic severity (e.g., severe classical phenotypes or attenuated non-classical phenotypes) resulting in reduced GALNS enzymatic activity relative to a healthy baseline.

A polynucleotide refers to and includes a polymer of two or more nucleosides each joined by an inter-nucleoside linkage. An exogenous polynucleotide generally refers to and includes a polynucleotide that originates from or is derived from a source outside a given cell, tissue, or organism. For example, an exogenous polynucleotide generally refers to and includes a polynucleotide or poly-nucleobase sequence introduced into a host cell (such as a target CD34+ cell) regardless of whether an identical or homologous sequence naturally occurs within that host cell. Additional examples include deoxyribose polynucleotides having a cDNA nucleobase sequence encoding a GALNS enzyme, wherein the deoxyribose polynucleotide is integrated into the genome of a CD34+ cell.

Suitable locations within the genome CD34+ include genomic safe harbor sites. A genomic safe harbor site generally refers to and includes a locus within the genome where an exogenous polynucleotide can be integrated in a predictable and stable manner without causing adverse effects on the host cell. For example, a genomic safe harbor represents a site where integration does not result in insertional mutagenesis and/or disrupt the function of essential endogenous genes. Exemplary safe harbor sites include locations within the genome that are intragenic and within open chromatin. Non-limiting examples include the AAVS1 (Adeno-Associated Virus Site 1) locus located within the PPP1R12C gene on chromosome 19, the ROSA26 locus located on chromosome 3, and/or the CLYBL (Citrate Lyase Beta-Like) locus: located on chromosome 13. In certain embodiments, the safe harbor site is within AAVS1.

To achieve expression of the GALNS enzyme, the exogenous polynucleotide can also comprise a promoter sequence (e.g., located 5′ to the protein coding sequence) that initiates transcription of the protein coding sequence encoding the GALNS enzyme. Suitable promoters include, but are not limited to ubiquitous promoters (e.g., e.g., EF1alpha, CAG, PGK, UBC, and CMV). In certain instances, an endogenous promoter (e.g., the PPP1R12C promoter) can also be used to initiate transcription of the protein coding sequence. The exogenous polynucleotide can also include additional elements for the transcription and translation of the protein coding sequence. For example, a 5′ UTR located 5′ to the protein coding sequence, 3′ UTR located 3′ to the protein coding sequence, and a poly-A sequence located 3′ to the 3′ UTR. Exemplary poly-A sequences include but are not limited to, the bovine growth hormone (bGH) polyadenylation signal, the simian virus 40 (SV40) polyadenylation signal, the human growth hormone (hGH) polyadenylation signal, and the rabbit beta-globin (rBG) polyadenylation signal. Examples of UTR (untranslated region) sequences include, but are not limited to, the human alpha-globin UTR, the human beta-globin UTR, the Woodchuck Hepatitis Virus Posttranscriptional Regulatory Element (WPRE), the human EF1alpha UTR, or a combination thereof.

As exemplified in the Examples, the modified CD34+ cells described herein can be characterized by one or more (or all) of the following functional outcomes: (1) the modified CD34+ cell secretes the GALNS enzyme, (2) co-culturing of the modified CD34+ cell with GALNS-deficient cells (e.g., MPS IVA fibroblasts) results in an increase in the GALNS activity in the MPS IVA fibroblasts, (3) the modified CD34+ cell is capable of forming colony-forming units (CFUs) (e.g., one or more of CFU-GEMM, CFU-GM, BFU-E, CFU-G, and CFU-M), (4) co-culturing of the modified CD34+ cell with GALNS-deficient cells (e.g., MPS IVA fibroblasts) results in a decrease in lysosomal mass, (5) co-culturing of the modified CD34+ cell with GALNS-deficient cells (e.g., MPS IVA fibroblasts) results in reduction of GAG (e.g., mono-KS) accumulation, (6) co-culturing of the modified CD34+ cell with GALNS-deficient cells (e.g., MPS IVA fibroblasts) results in a decrease in reactive oxygen species, (7) co-culturing of the modified CD34+ cell with GALNS-deficient cells (e.g., MPS IVA fibroblasts) results in a decrease in cell death (e.g., apoptosis), and/or (8) co-culturing of the modified CD34+ cell with GALNS-deficient cells (e.g., MPS IVA fibroblasts) results in reduced pro-inflammatory biomarkers (e.g., interleukin-1 beta (IL1b) or interleukin-6 beta (IL6b)). Such outcomes and non-limiting examples of assays for their determination/confirmation are described and exemplified in the Examples.

### Methods of Use

The modified CD34+ cells described herein are, in certain instances, useful in increasing the amount of GALNS enzyme and GALNS enzyme activity. Uses for the mRNA polynucleotides described herein include, without limitation: (1) methods of increasing an amount of GALNS and GALNS activity (e.g., in an environment), (2) methods of increasing GALNS activity of GALNS-deficient cells, (3) methods of reducing glycosaminoglycan (GAG) accumulation in GALNS-deficient cells, (4) method of reducing lysosomal mass in GAL-deficient cells, (5) methods of reducing metabolic stress in GALNS-deficient cells, and/or (6) reducing pro-inflammatory proteins within a population of GALNS-deficient cells. As exemplified in the Examples, such uses can generally be achieved by co-localizing modified CD34+ cells with GALNS-deficient cells.

Co-localizing generally refers to and includes the spatial overlap or concurrent presence of at least two cells (e.g., modified CD34+ cells and GALNS-deficient cells) within a compartment. In certain instances, co-localization refers to and includes at least two cells in fluid communication with one another. As described herein, such co-localization or fluid communication facilitates, in certain instances, cross-correction, wherein the increased amount of functional GALNS enzyme secreted by the modified CD34+ cells (and functional benefit) is acquired by neighboring GALNS-deficient cells to increase intracellular and extracellular GALNS enzymatic activity. A compartment generally refers to and includes a defined biological, physiological, or artificial microenvironment that permits the co-localization (e.g., spatial localization), fluidic connection, or chemical communication between at least two cells (e.g., modified CD34+ cell population and GALNS-deficient cells).

Examples of increasing an amount of GALNS activity includes, but is not limited to, extracellular GALNS activity, intracellular GALNS activity, or both. Non-limiting examples of how GALNS activity can be measured are described herein and exemplified in the Examples. In certain instances, a reduction in lysosomal mass within GALNS-deficient cells can be a proxy measurement for increased GALNS activity.

Examples of reducing glycosaminoglycan (GAG) accumulation in GALNS-deficient cells include, but are not limited to, instance where the GAG comprises keratan sulfate (KS) (e.g., mono-KS). Non-limiting examples of how KS accumulation can be measured are described and exemplified in the Examples, for example, by using liquid chromatography-tandem mass spectrometry (LC-MS/MS).

Examples of reducing metabolic stress include, but are not limited to (1) a reduction of mitochondrial reactive oxygen species within a population of GALNS-deficient cells, (2) a reduction of global reactive oxygen species within a population of GALNS-deficient cells, and/or (3) a reduction in mitochondrial mass, and reduced cell death. Non-limiting examples of how each can be measured are described and exemplified in the Examples.

Non-limiting examples of reducing pro-inflammatory proteins within a population of GALNS-deficient cells include a reduction in the pro-inflammatory biomarkers comprise interleukin-1 beta (IL1b) or interleukin-6 beta (IL6b). Examples of how pro-inflammatory biomarkers can be measured include ELISA and/or RT-PCT, and are described and exemplified in the Examples.

The modified CD34+ cells described herein are, in certain instances, also useful in increasing the amount of GALNS enzyme and/or GALNS enzyme activity in an individual having a GALNS deficiency, wherein the methods comprise administering the modified CD34+ cells to the individual. Examples of a GALNS deficiency include MPS IVA. In such instances, the modified CD34+ cells can be engineered from autologous CD34+ cell taken from the individual.

Exemplary route of administration includes, but is not limited to, intravenous (IV) administration. IV administration generally refers to and includes the introduction of a composition directly the venous circulation of an individual.

### Methods of Manufacture

Also provided herein are methods of making the modified CD34+ cells that express GALNS. As described in the Examples, such methods generally include cutting the genome of a CD34+ cell and inserting an exogenous polynucleotide (e.g., by providing the cell with the exogenous polynucleotide) having a protein coding sequence that encodes the GALNS enzyme. For example, a targetable gene editing system can be used to generate a cut site, and the exogenous polynucleotide can be provided, wherein the exogenous polynucleotide is inserted into the genome at the cut site. A targetable gene editing system generally refers to and includes any engineered molecular machinery capable of recognizing, binding to, and cutting a specific, predefined target sequence within a genome. Such systems generally comprise a sequence-specific DNA-targeting element (e.g., a guide RNA or a programmable DNA-binding protein domain) physically or functionally coupled to a nuclease (e.g., a nickase) or nuclease effector domain. Non-limiting examples of targetable gene editing systems include CRISPR/Cas (Clustered Regularly Interspaced Short Palindromic Repeats/CRISPR-associated) systems, Zinc Finger Nucleases (ZFNs), Transcription Activator-Like Effector Nucleases (TALENs), and engineered meganucleases.

Suitable targetable gene editing systems include, but are not limited to, CRISPR-Cas gene editing systems. CRISPR-Cas gene editing systems generally refer to and include one or more RNA-guided nucleases derived from Clustered Regularly Interspaced Short Palindromic Repeats (CRISPR) adaptive immune system of bacteria and archaea, or any synthetic, optimized, or engineered variations thereof. In certain instances, the CRISPR-Cas system comprises one or more CRISPR-Cas complexes comprising a CRISPR-associated (Cas) effector nuclease and a guide RNA. The Cas effector nuclease can comprise a wild-type Cas nuclease or a modified Cas nickase (nCas). Cas effector nucleases are known in the art and non-limiting examples of Cas effector nucleases include Cas9, Cas12a (Cpf1), Cas12b, Cas12e, Cas13, and orthologs or engineered variants thereof. Generally, upon hybridization of the guide RNA (via the spacer sequence) to a complementary target protospacer sequence (e.g., within a genomic safe harbor locus), the Cas effector protein induces a targeted genetic modification, such as a double-strand break or a single-strand nick, to facilitate the integration of the exogenous GALNS-encoding polynucleotide.

Examples of CRISPR-Cas gene editing systems include, but are not limited to one or more CRISPR-Cas complexes comprising a CRISPR-Cas nuclease and a guide RNA (gRNA) that targets the first nuclease to a first site within the cut site. For example, a CRISPR-Cas gene editing system can comprise: (i) a first CRISPR-Cas complex comprising a first CRISPR-Cas nuclease and a first guide RNA (gRNA) that targets the first nuclease to a first site within the cut site; (ii) a second CRISPR-Cas complex comprising a second CRISPR-Cas nuclease and a second guide RNA (gRNA) that targets the second nuclease to a second site within the cut site. In certain instances, the CRISPR-Cas nuclease is a nickase (nCas). The CRISPR-Cas gene editing systems can be provided to a cell as RNP complexes in the form of one or more polynucleotides encoding the CRISPR-Cas nuclease and one or more guides.

As described herein, the site at which the exogenous polynucleotide is integrated can be within a genomic safe harbor site, wherein the genomic safe harbor site is located within an intragenic site and open chromatin. The exogenous polynucleotide can be provided to the CD34+ cell via transfecting or transducing the CD34+ cell with the exogenous polynucleotide. Transfection generally refers to and includes the process of introducing one or more exogenous nucleic acids (e.g., mRNA) into a cell by non-viral means. Transfection can generally include both carrier-based or chemical methods (e.g., using lipid nanoparticles, cationic lipids, or polymers) and physical methods (e.g., electroporation or sonoporation). In certain instances, transfection includes ex vivo transfection. Non-limiting examples of carriers used for transfection include LNPs. Transduction generally refers to and includes the introduction of an exogenous polynucleotide (e.g., a donor template or a sequence encoding a gene-editing system) into a target cell utilizing a viral vector. Suitable viral vectors include, but are not limited to, recombinant adeno-associated viral (rAAV) vectors, lentiviral vectors, retroviral vectors, adenoviral vectors, and herpes simplex viral vectors. To facilitate insertion/integration, the exogenous polynucleotide can include a 5′ and 3′ homologous recombination (HR) sequence.

### ADDITIONAL DEFINITIONS

An “individual” or “subject” are interchangeable and generally refers to and includes any human or non-human animal to whom a composition or method can be administered, including humans, non-human primates, and other mammals.

The term sequence identity or percent (%) sequence identity refers to the percentage of identical nucleotides or amino acids in a sequence compared to a reference sequence, when the sequences are aligned using a sequence alignment program such as BLAST or ClustalW, with default parameters. Sequence identity is calculated by comparing the aligned positions in the two sequences and dividing the number of identical residues by the total number of positions compared, multiplied by 100%.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “up to,” “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

In some instances, a value is “about” a recited value if the value is within ±10% of the recited value. In some instances, disclosure of “about” a recited value includes disclosure of a value that is within ±5% of the recited value.

The term “comprise” and variations thereof, such as, “comprises” and “comprising” are to be construed in an open, inclusive sense, that is, as “including, but not limited to.” It is also to be understood that disclosure of “comprising” is also intended to include disclosures of “consisting essentially of” and “consisting of” the same recited elements. Generally, “consisting essentially of” is to be construed as partially open, being directed to those elements set forth and to those other elements that do not materially affect the basic and novel characteristics of the invention described. Generally, “consisting of” means that the claim is directed only to the specified elements.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

### EXEMPLARY EMBODIMENTS

In addition to those already described herein, non-limiting examples of embodiments provided and described herein also include, but are not limited to, the following enumerated embodiments:

Embodiment 1: A modified CD34 positive (CD34+) cell comprising an exogenous polynucleotide, wherein:
(i) the exogenous polynucleotide comprises a protein coding sequence that encodes a N-acetylgalactosamine-6-sulfatase (GALNS) enzyme; and
(ii) the exogenous polynucleotide is integrated into the genome of the CD34+ cell.

Embodiment 2: The modified CD34+ cell of embodiment 1, wherein the GALNS enzyme comprises an amino acid sequence having at least 90% sequence identity to SEQ ID NO: 1.

Embodiment 3: The modified CD34+ cell of embodiment 1 or embodiment 2, wherein the GALNS enzyme hydrolyzes a sulfate group from a 4-methylumbelliferyl-β-D-galactopyranoside-6-sulfate (4MU-Gal-6S) substrate (e.g., in a fluorometric assay).

Embodiment 4: The modified CD34+ cell of any one of embodiments 1-3, wherein the exogenous polynucleotide further comprises a promoter sequence, wherein the promoter sequence initiates transcription of the protein coding sequence encoding the GALNS enzyme.

Embodiment 5: The modified CD34+ cell of any one of embodiments 1-4, wherein the CD34+ is a CD34+ stem cell.

Embodiment 6: The modified CD34+ cell of embodiment 5, wherein the CD34+ stem cell is a bone marrow CD34+ stem cell.

Embodiment 7: The modified CD34+ cell of any one of embodiments 1-6, wherein the exogenous polynucleotide is integrated into a genomic safe harbor site within the genome.

Embodiment 8: The modified CD34+ cell of embodiment 7, wherein the genomic safe harbor site is located within an intragenic site and open chromatin.

Embodiment 9: The modified CD34+ cell of any one of embodiments 1-8, wherein the modified CD34+ cell secretes the GALNS enzyme.

Embodiment 10: The modified CD34+ cell of any one of embodiments 1-10, wherein co-culturing of the modified CD34+ cell with MPS IVA fibroblasts results in an increase in the GALNS activity in the MPS IVA fibroblasts.

Embodiment 11: The modified CD34+ cell of embodiment 10, wherein the GALNS activity is extracellular GALNS activity, intracellular GALNS activity, or both extracellular GALNS activity and intracellular GALNS activity.

Embodiment 12: The modified CD34+ cell of any one of embodiments 1-11, wherein the modified CD34+ cell is capable of forming colony-forming units (CFUs), wherein the CFUs comprise one or more of CFU-GEMM, CFU-GM, BFU-E, CFU-G, and CFU-M.

Embodiment 13: Modified CD34+ positive cells comprising a plurality of the modified CD34+ cells of any one of embodiments 1-12.

Embodiment 14: A method of increasing GALNS activity of GALNS-deficient cells, the method comprising: co-localizing the modified CD34+ cells of embodiment 12 with the GALNS-deficient cells.

Embodiment 15: The method of embodiment 14, wherein the method results in increased GALNS activity.

Embodiment 16: The method of embodiment 15, wherein the GALNS activity is extracellular GALNS activity, intracellular GALNS activity, or both extracellular GALNS activity and intracellular GALNS activity.

Embodiment 17: A method of reducing glycosaminoglycan (GAG) accumulation in GALNS-deficient cells, the method comprising: co-localizing the modified CD34+ cells of embodiment 12 with the GALNS-deficient cells.

Embodiment 18: The method of embodiment 17, wherein the method results in a reduction in GAG accumulation in the GALNS-deficient cells.

Embodiment 19: The method of embodiment 18, wherein the GAG comprises keratan sulfate (KS).

Embodiment 20: The method of embodiment 19, wherein the KS is mono-KS.

Embodiment 21: A method of reducing metabolic stress in GALNS-deficient cells, the method comprising: co-localizing the modified CD34+ cells of embodiment 12 with the GALNS-deficient cells.

Embodiment 22: The method of embodiment 21, wherein the method results in a reduction of metabolic stress within the GALNS-deficient cells.

Embodiment 23: The method of embodiment 22, wherein the method results in a reduction of mitochondrial reactive oxygen species within the GALNS-deficient cells.

Embodiment 24: The method of embodiment 22, wherein the method results in a reduction of mitochondrial mass of the GALNS-deficient cells.

Embodiment 25: The method of embodiment 22, wherein the method results in a reduction in cell death of the GALNS-deficient cells.

Embodiment 26: A method of reducing pro-inflammatory proteins within GALNS-deficient cells, the method comprising: co-localizing the modified CD34+ cells of embodiment 12 with the GALNS-deficient cells.

Embodiment 27: The method of embodiment 26, wherein the method results in a reduction of pro-inflammatory biomarkers within the GALNS-deficient cells.

Embodiment 28: The method of embodiment 27, wherein the pro-inflammatory biomarkers comprise interleukin-1 beta (IL1b) or interleukin-6 beta (IL6b).

Embodiment 29: A method of making a modified CD34+ cell, the method comprising: cutting the genome of a CD34+ cell using a targetable gene editing system to generate a cut site; and providing an exogenous polynucleotide to the CD34+ cell, wherein the exogenous polynucleotide comprises a protein coding sequence that encodes a N-acetylgalactosamine-6-sulfatase (GALNS) enzyme and is inserted into the genome at the cut site.

Embodiment 30: The method of embodiment 29, wherein the targetable gene editing system comprises a CRISPR-Cas gene editing system.

Embodiment 31: The method of embodiment 30, wherein the CRISPR-Cas gene editing system comprises:
(i) a first CRISPR-Cas complex comprising a first CRISPR-Cas nuclease and a first guide RNA (gRNA) that targets the first nuclease to a first site within the cut site;
(ii) a second CRISPR-Cas complex comprising a second CRISPR-Cas nuclease and a second guide RNA (gRNA) that targets the second nuclease to a second site within the cut site.

Embodiment 32: The method of any one of embodiments 29-31, wherein the cut site is within a genomic safe harbor site of the genome.

Embodiment 33: The method of embodiment 32, wherein the genomic safe harbor site is located within an intragenic site and open chromatin.

Embodiment 34: The method of any one of embodiments 29-33, wherein providing an exogenous polynucleotide to the CD34+ cell comprises transfecting or transducing the CD34+ cell with the exogenous polynucleotide.

Embodiment 35: A method of increasing an amount of GALNS enzyme in an individual having a GALNS deficiency, the method comprising: administering the modified CD34+ cells of embodiment 12 to the individual.

Embodiment 36: The method of embodiment 35, wherein the method results in an increased amount of GALNS in the individual.

Embodiment 37: The method of embodiment 35 or embodiment 36, wherein the GALNS deficiency is MPS IVA.

### EXAMPLES

### Example 1

### 1.1 Summary

Mucopolysaccharidosis (MPS) IVA is a bone-affecting lysosomal storage disease (LSD) caused by impaired degradation of the glycosaminoglycans (GAGs), keratan sulfate (KS) and chondroitin 6-sulfate (C6S) due to deficient N-acetylgalactosamine-6-sulfatase (GALNS) enzyme activity. Previously, a CRISPR/nCas9-based gene therapy (GT) was developed and validated to insert an expression cassette at the AAVS1 and ROSA26 locus in human MPS IVA fibroblasts and MPS IVA mice, respectively. In this study, the approach was extended to evaluate the effectiveness of the CRISPR/nCas9-based GT in editing human CD34+ cells to mediate cross-correction of MPS IVA fibroblasts. CD34+ cells were electroporated with the CRISPR/nCas9 system targeting the AAVS1 locus. The nCas9-mediated on-target, donor template insertion, and stemness of CRISPR/nCas-edited CD34+ cells were evaluated. Besides, MPS IVA fibroblasts were co-cultured with CRISPR/nCas-edited CD34+ cells to assess cross-correction. CRISPR/nCas9-based gene editing did not affect the stemness of CD34+ cells while leading to supraphysiological levels of the GALNS enzyme. Upon co-culture, MPS IVA fibroblasts displayed a significant increase in the GALNS enzyme activity along with lysosomal mass reduction, pro-oxidant profile amelioration, mitochondrial mass recovery, and pro-apoptotic and pro-inflammatory profile improvement. These results show the potential of the CRISPR/nCas9-based GT to edit CD34+ cells to mediate cross-correction.

Mucopolysaccharidosis (MPS) IVA (OMIM:) is a genetic lysosomal storage disease (LSD) caused by a deficiency in the N-acetylgalactosamine-6-sulfatase (GALNS) enzyme that leads to the lysosomal accumulation of the glycosaminoglycans (GAGs), keratan sulfate (KS) and chondroitin 6-sulfate (C6S). The accumulation of KS and C6S disrupts the function of chondrocytes in the growth plates, impairs cartilage matrix development, and inhibits bone matrix mineralization. The resultant incomplete or defective endochondral ossification and successive imbalance of growth in MPS IVA patients cause unique skeletal symptoms such as prominent forehead, abnormal face with a large mandible, disproportionate short-trunk dwarfism with short neck, cervical spine instability with odontoid hypoplasia, cervical spinal cord compression, pectus carinatum, tracheal deviation and obstruction, restrictive lung, flaring of the rib cage, kyphoscoliosis, platyspondyly, hip dysplasia with coxa valga, genu valgum, hypermobile joints, waddling gait, and pes planus. The degree of imbalance of growth in bone and other organs and tissues largely contributes to unique skeletal dysplasia and clinical severity, including narrowing trachea. Currently, an enzyme replacement therapy (ERT) is approved to treat MPS IVA patients; nevertheless, ERT has several limitations: i) a limited impact on skeletal dysplasia, ii) infused enzymes are unstable and taken up mainly by the liver and spleen in rapid clearance, iii) most patients with ERT produce antibodies against the enzyme developing immune reactions, and iv) ERT costs $500,000/year/kg in the USA. Allogeneic hematopoietic stem cell (HSC) transplantation is a therapeutic option available for patients with MPS IVA; nevertheless, the risk of development of graft versus host disease (GVHD) remains a significant concern with a limited bone impact and human leukocyte antigens (HLA)-matched donor. Other approaches, including pharmacological chaperones (PC) and substrate degradation therapy (SDT), have been tested and could turn into novel therapies shortly.

Classical- and CRISPR/Cas9-based gene therapy (GT) alternatives have been tested using in vitro and in vivo models of MPS IVA with encouraging results. Regarding the CRISPR/Cas9 system, a novel strategy using Cas9 Nickase (nCas9) was reported to introduce double nicking at the AAVS1 locus to mediate homologous recombination of an expression cassette carrying a wild-type (WT) cDNA of GALNS. In vitro data showed a significant phenotype recovery in MPS IVA fibroblasts carrying different mutations in the GALNS gene. Likewise, when evaluating the CRISPR/nCas9 system in MPS IVA mice, a significant improvement in the monosulfated KS levels was achieved upon direct infusion using iron oxide nanoparticles as non-viral vectors. Despite these promising findings, direct injection of the CRISPR/nCas9 system partially rescued bone lesions, suggesting that novel bone-impact approaches must be explored.

In 2019, Gomez-Ospina et al. successfully implemented an ex vivo-based CRISPR/Cas9 GT in MPS I mice. The CRISPR/Cas9 system was targeted to the human CCR5 locus in hematopoietic stem progenitor cells (HSPCs) to insert an expression cassette containing IDUA cDNA, which is impaired in MPS I. CRISPR/Cas9-edited HSPCs showed stable gene insertion while preserving stemness and engraftment potential. Most interestingly, CRISPR/Cas9-edited HSPCs normalized femur, zygomatic, and skull bone thickness, suggesting that ex vivo CRISPR/Cas9-mediated GT could be a potential alternative for treating bone manifestations in MPS. Consequently, using the CRISPR/Cas9 system to insert expression cassettes in MPS IVA patient-derived HSPCs could represent a novel alternative to overcome GVDH while inducing the expression of the functional GALNS enzyme, which can correct bone pathology and systemic manifestations of the disease.

Ex vivo lentiviral (LV) GT was recently tested in MPS IVA mice by Celik et al. Interestingly, while ex vivo LV GT resulted in higher GALNS expression in plasma and tissues than in studies using the CRISPR/nCas9 system, both LV- and CRISPR/nCas9-mediated GT partially recovered the bone pathology. Although it was not evaluated by Celik et al., LV-based GT raises several concerns due to the LV random integration, which could increase the potential risk of oncogenesis. Early ex vivo LV-mediated GT in two patients suffering from X-linked chronic granulomatosis resulted in myelodysplasia or acute myeloid leukemia due to insertional activation of oncogenes. Clinical trials recently observed similar hematological findings for cerebral adrenoleukodystrophy with ex-vivo LV GT. These unwanted effects can be significantly reduced using the CRISPR/Cas9 system due to the high precision during gene insertion and the elimination of off-target effects when using nCas9.

In light of this evidence, the effectiveness of the CRISPR/nCas9 system was first assessed in editing CD34+ hematopoietic stem cells (HSCs) and proving the cross-correction of MPS IVA fibroblasts.

### 1.2 CRISPR/nCas9-Based Genome Editing System Efficiently Preserves the Self-Renewal and Differentiation Potential of CD34+ Cells

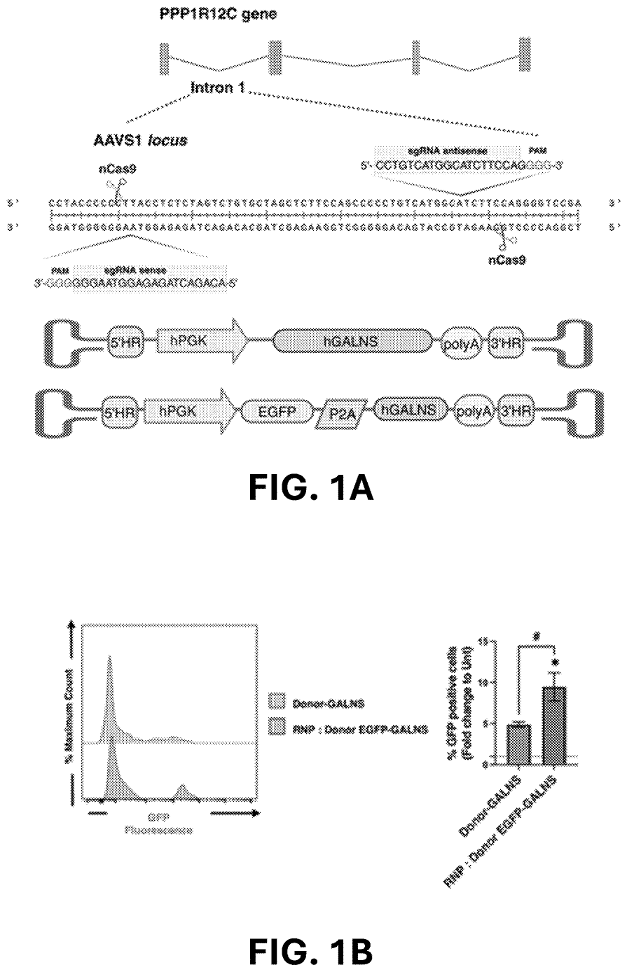

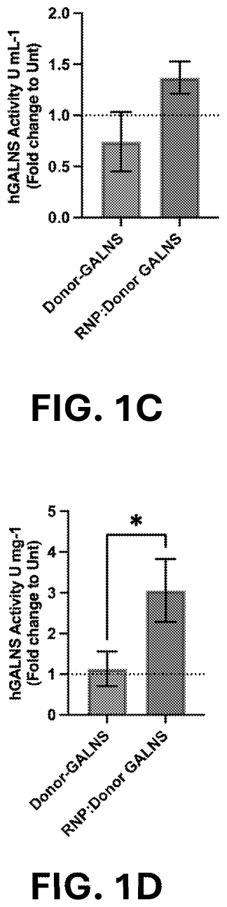

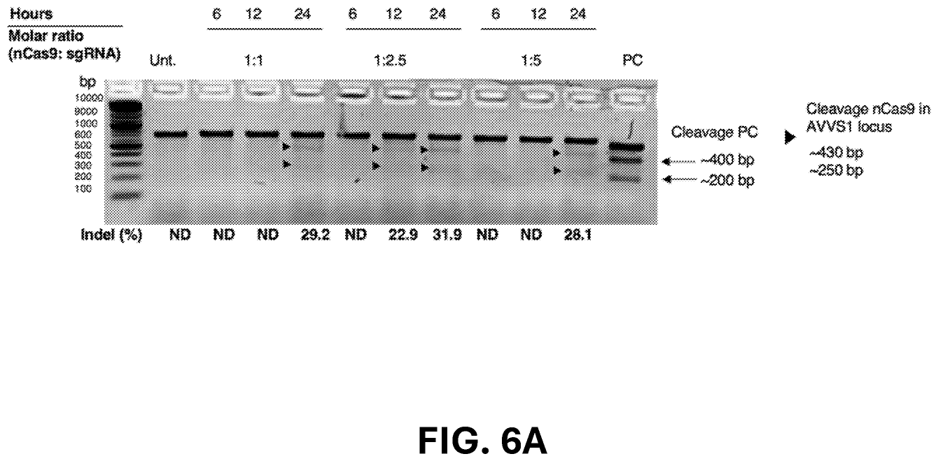

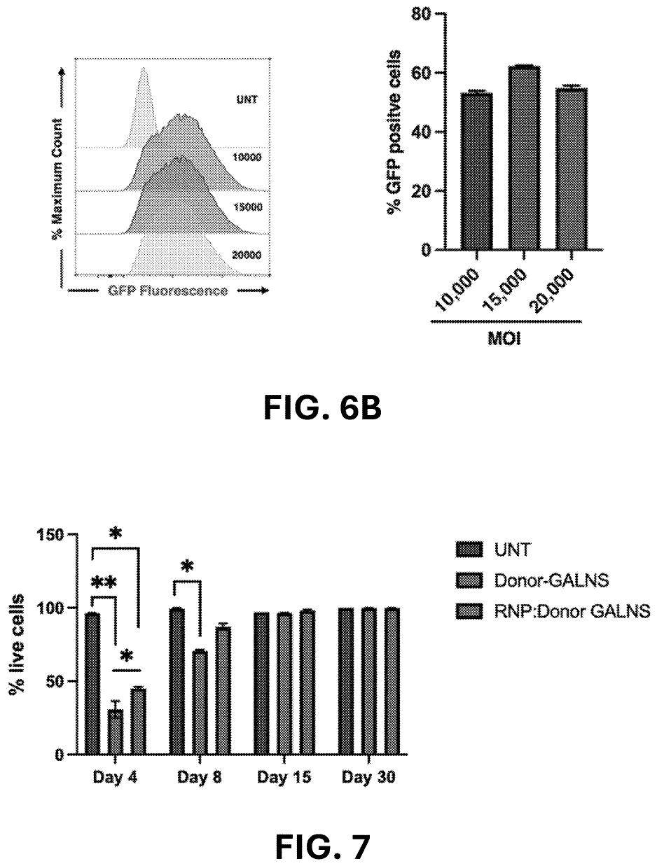

In this study, an RNP-based CRISPR/nCas9 system was evaluated to mediate the homologous recombination (HR) of the expression cassette using AAV6 as a viral vector (FIG. 1A). Initially, HEK-293 cells were transfected with the RNP-based CRISPR/nCas9 system at various molar ratios (nCas9:sgRNA) over hours to optimize nCas9 cutting. nCas9-dependent DNA cutting was determined by a mismatch recognizing strategy using the T7 endonuclease assay. Under experimental conditions, an on-target efficiency was found at a 1:2.5 molar ratio (FIG. 6A). Then, the transduction efficiency of the AAV6-donor template was tested at different MOIs. HEK-293 cells were transduced with AAV6-EGFP/GALNS, and the percentage of cells expressing the reporter GFP was analyzed by flow cytometry (FIG. 6B). A multiplicity of infection (MOI) exhibited the highest transduction rate as observed via EGFP expression.

Following optimization experiments, CD34+ cells were edited using the RNP complex and AVV6-Donor-EGFP at a 1:2.5 molar ratio and MOI, respectively, and analyzed after days of culturing. As shown in FIG. 1B, AAV6-Donor-EGFP transduction in the absence of the RNP complex resulted in a fold increase in the EGFP expression compared to untreated CD34+ cells. When CD34+ cells were edited by electroporating the RNP complex followed by AAV6 transduction, a fold increase in EGFP-positive cells was observed relative to untreated CD34+ cells days post-editing, suggesting that the RNP complex led to a better expression of EGFP by the integration of the expression cassette in CD34+ cells. Assessing the therapeutic effectiveness of RNP-based CRISPR/nCas9 system and AAV6-Donor transduction. Genome editing efficiency was further evaluated by measuring GALNS enzyme activity in CRISPR/nCas9-edited CD34+ cells (FIG. 1C, FIG. 1D). While AAV6-Donor transduction alone resulted in a fold increase in the extra- and intra-cellular GALNS enzyme activity, respectively, the RNP complex electroporation followed by AAV6-Donor transduction notably increased the extra- and intra-cellular GALNS enzyme activity up to a fold increase in CD34+ cells (FIG. 1C, FIG. 1D).

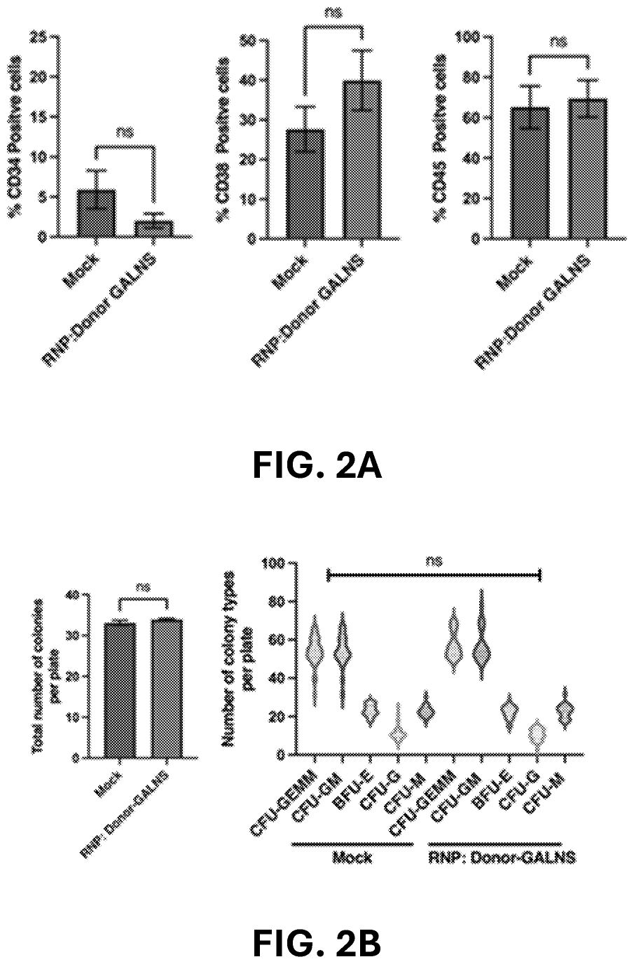

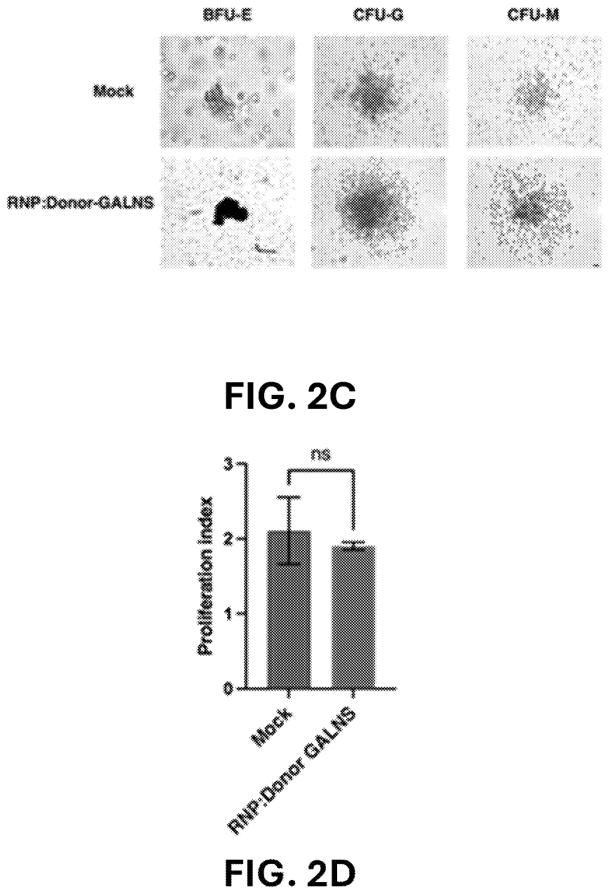

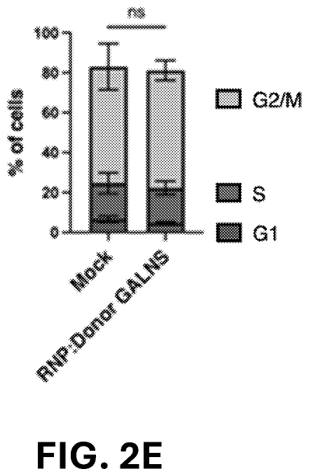

To evaluate the impact of the RNP-based CRISPR/nCas9 system on the stemness of CD34+ cells, the stem properties (differentiation and self-renewal) of CRISPR/nCas9-edited CD34+ cells were assessed days post-editing. First, the frequency of CD markers and the expression profile of primitive hematopoietic progenitor cells (CD34+, CD38+, and CD45+) were examined by flow cytometry (FIG. 2A). No significant changes were found in the frequencies of the CD34+, CD38+, and CD45+ markers in CRISPR/nCas9-edited CD34+ cells compared to untransfected cells days post-editing (FIG. 2A). Next, the differentiation potential of CRISPR/nCas9-edited CD34+ cells was evaluated by assessing colony-forming unit (CFU) through flow cytometry and microscopy assessment. No difference was observed in the number of colonies per plate or colony types between CRISPR/nCas9-edited CD34+ cells and untransfected cells days post-editing using either method (FIG. 2B, FIG. 2C). Through flow cytometry analysis using the differentiation markers CD15, CD14, and CD235a, it was identified that CRISPR/nCas9-edited CD34+ cells retained the capacity to differentiate into multipotent progenitor cells, including the CFU-GEMM (multipotential colony-forming cells), and committed progenitor cells, such as CFU-GM (Colony Forming Unit-Granulocyte-Macrophage) and BFU-E (Erythroid Burst-Forming Units). Additionally, differentiation into mature multilineage colonies, such as CFU-G (Granulocyte Progenitor Cells) and CFU-M (Monocytic Progenitor Cells) (FIG. 2B) was detected, suggesting that the CRISPR/nCas9 system does not affect the stemness of CD34+ cells.

Using flow cytometry, the proliferation ability and cell cycle preservation in CRISPR/nCas9-edited CD34+ cells were also assessed. No significant differences were observed in the proliferation index (FIG. 2D) or cell cycle frequency (FIG. 2E) days post-editing. Although a decrease in cell viability was observed on day post-treatment, CRISPR/nCas9-edited CD34+ cells exhibited a faster recovery overtime compared to CD34+ cells transduced with AAV6-Donor alone (FIG. 7), highlighting that AAV6 alone can increase cell toxicity. These results demonstrate that the RNP-based CRISPR/nCas9 system successfully increases GALNS enzyme levels in CD34+ cells, preserving their stemness after long-term post-editing.

### 1.3 Therapeutic Efficacy of CRISPR/nCas9-Edited HSCs in MPS IVA Fibroblasts Co-Culture

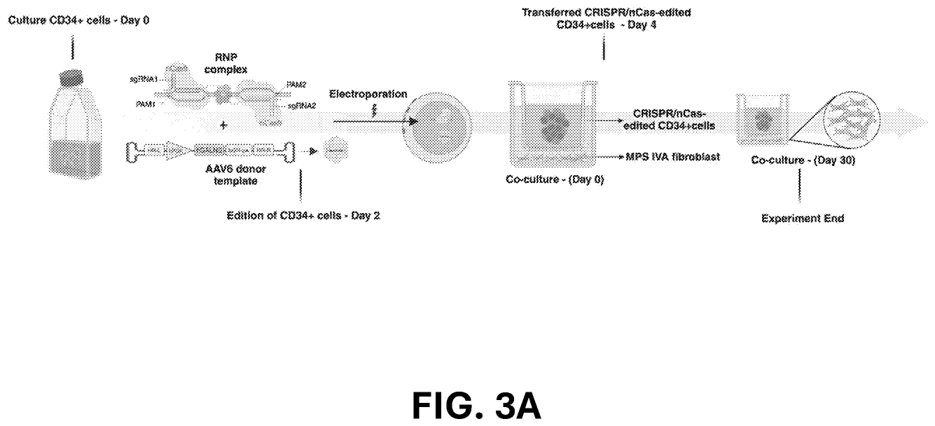

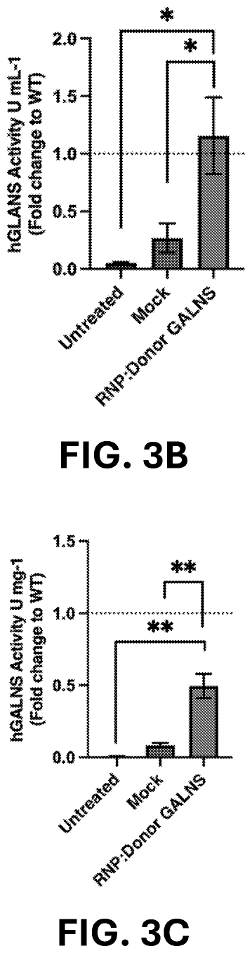

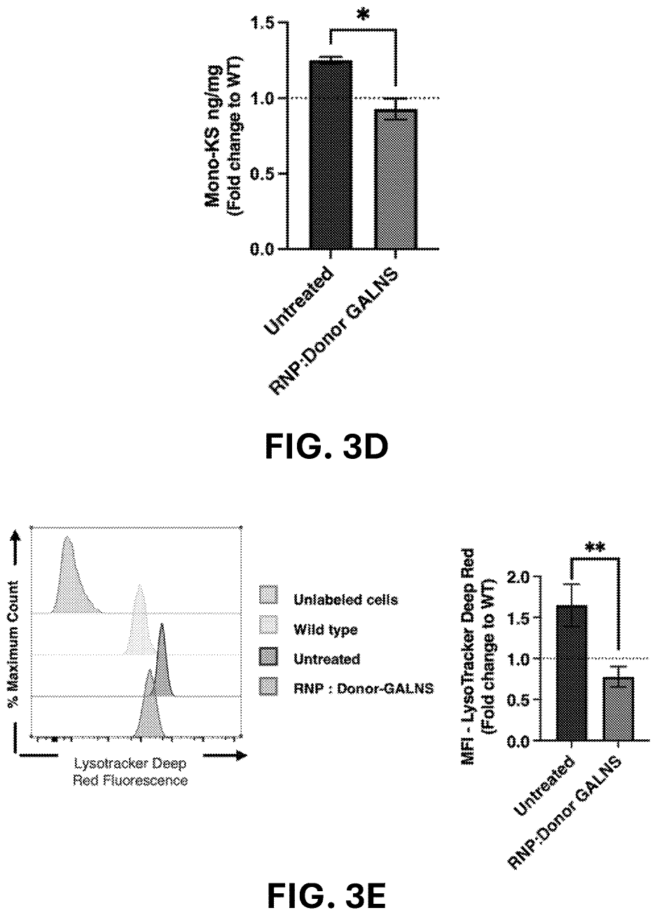

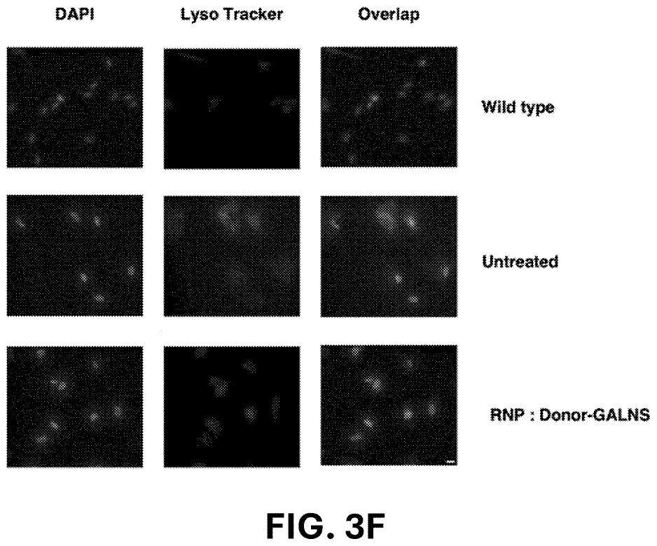

The phenotype recovery was evaluated by co-culturing MPS IVA fibroblasts with CRISPR/nCas9-edited CD34+ cells using a transwell-mediated approach (FIG. 3A). While untreated MPS IVA fibroblasts exhibited no detectable GALNS enzyme activity (FIG. 3B, FIG. 3C), co-culture of MPS IVA fibroblasts with mock-treated CD34+ cells increased the extracellular GALNS enzyme activity (WT levels). Conversely, co-culture of MPS IVA fibroblasts with CRISPR/nCas9-edited CD34+ cells led to significant extracellular GALNS enzyme activity, reaching levels comparable to WT fibroblasts (FIG. 3B). Similarly, intracellular GALNS activity in MPS IVA fibroblasts co-cultured with CRISPR/nCas9-edited CD34+ cells reached up to of WT levels (FIG. 3C). In contrast, co-culture with mock-treated CD34+ cells resulted in only an increase of WT levels in intracellular GALNS enzyme activity (FIG. 3C). These results strongly suggest that CRISPR/nCas9-edited CD34+ cells overexpress bioactive GALNS enzyme, which is uptaken by MPS IVA fibroblasts.

Next, the impact of these findings on classical pathological biomarkers of MPS IVA was evaluated. While untreated MPS IVA fibroblasts showed an increase in the mono-sulfated KS levels compared to WT fibroblasts, MPS IVA fibroblasts co-cultured with CRISPR/nCas9-edited CD34+ cells led to normalization of mono-sulfated KS levels to WT (FIG. 3D). Similarly, when evaluating the lysosomal mass via flow cytometry, untreated MPS IVA fibroblasts showed up to a fold increase in the lysosomal mass compared to WT fibroblasts. Upon co-culture with CRISPR/nCas9-edited CD34+ cells, the lysosomal mass in MPS IVA fibroblasts was normalized to WT levels (FIG. 3E). Similar findings were noticed in epifluorescence microscopy assays (FIG. 3F). These findings strongly suggest that CRISPR/nCas9-edited CD34+ cells can positively impact the lysosomal mass in MPS IVA fibroblasts through cross-correction.

### 1.4 Reduction of Oxidative Stress in MPS IVA Fibroblasts

Alterations in cellular homeostasis and oxidative stress have been identified as critical factors in the pathogenesis of various MPS. Previously, an improved oxidative profile, particularly in mitochondrial-related oxidative stress (mt-ROS), in MPS IVA and GM2 gangliosidoses fibroblasts upon CRISPR/nCas9 treatment was reported. To assess the impact of CRISPR/nCas9-edited CD34+ cells on the oxidative profile of MPS IVA fibroblasts, the global- and mt-ROS were quantified via flow cytometry after one month. Menadione (vitamin K3), known to induce cell death through ROS-dependent mechanisms, was included as a positive control in these experiments.

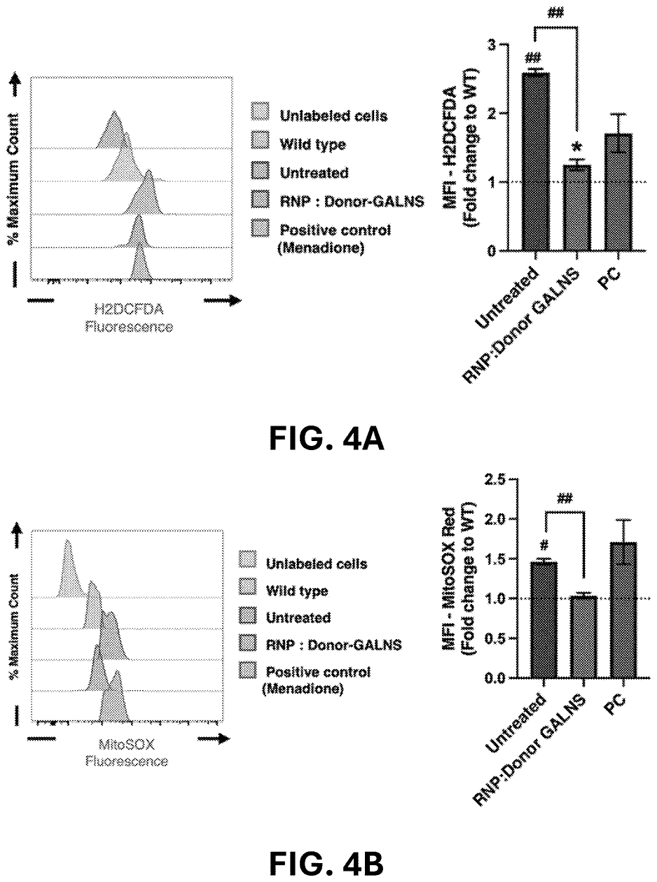

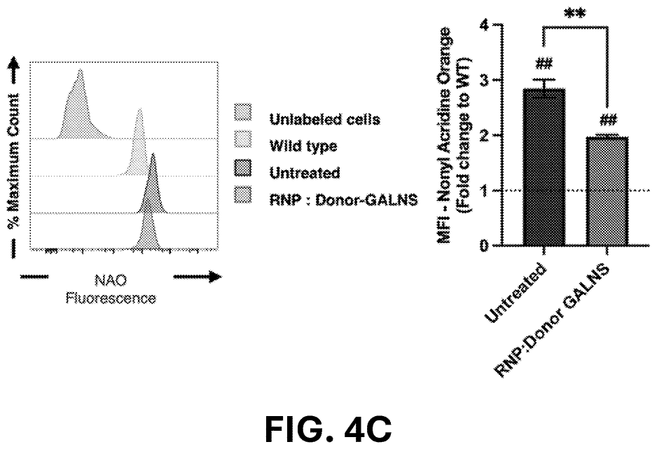

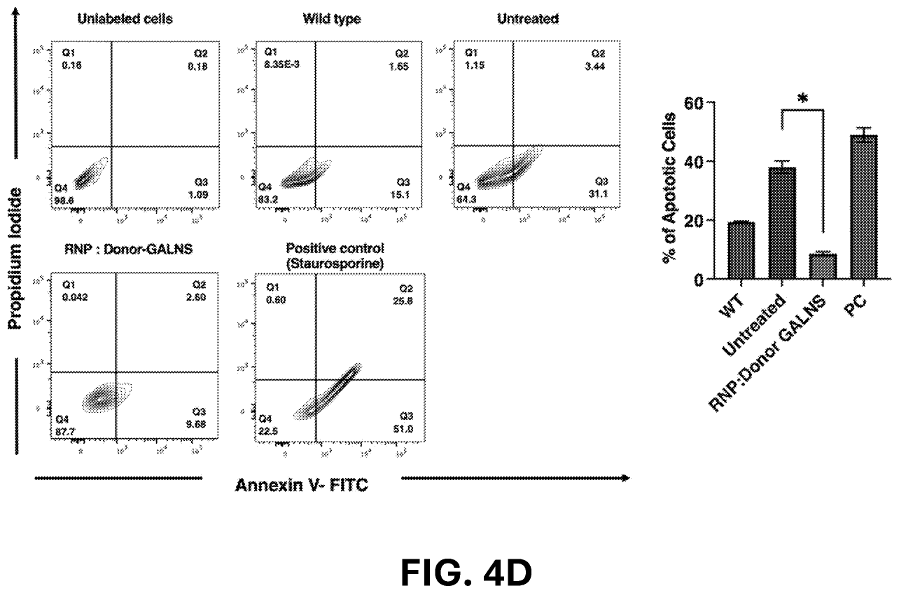

Untreated MPS IVA fibroblasts were characterized by a significant increase in the global ROS compared to WT fibroblasts (FIG. 4A). Although co-culture of CRISPR/nCas9-edited CD34+ cells and MPS IVA fibroblasts resulted in a significant reduction in global ROS levels in MPS IVA fibroblasts, they remained significantly higher than those observed in WT fibroblasts. Regarding mt-ROS, untreated MPS IVA fibroblasts showed an increase compared to WT fibroblasts (FIG. 4B). Conversely to global-ROS levels, co-culture of MPS IVA fibroblasts and CRISPR/nCas9-edited CD34+ cells resulted in mt-ROS normalization (FIG. 4B).

Growing evidence supports increased mitochondrial mass in some LSDs. To assess the mitochondrial mass in MPS IVA fibroblasts and the impact of the CRISPR/nCas9-based GT approach, the mitochondrial mass was monitored using Nonyl Acridine Orange (NAO) via flow cytometry. Untreated MPS IVA fibroblasts exhibited an increase in mitochondrial mass compared to WT fibroblasts (FIG. 4C). Upon co-culture with CRISPR/nCas9-edited CD34+ cells, a reduction in the mitochondrial mass was observed in co-cultured MPS IVA fibroblasts compared to untreated MPS IVA fibroblasts (FIG. 4C), suggesting amelioration in the mitochondrial mass of MPS IVA fibroblasts.

An increased mitochondrial-related apoptosis ratio was recently reported in MPS IVA fibroblasts. Apoptosis levels were further tested using a flow cytometry approach to assess the effect of co-culturing MPS IVA fibroblasts with CRISPR/nCas9-edited CD34+ cells. Staurosporine induces cell death via intrinsic apoptotic pathways, and it was included as a positive control for these experiments. While untreated MPS IVA fibroblasts exhibited increased spontaneous apoptosis, WT fibroblasts showed a basal apoptosis ratio (FIG. 4D). After one month of co-culture, a reduction in apoptosis in MPS IVA fibroblasts was observed, suggesting that GALNS enzyme supply via CRISPR/nCas9-edited CD34+ cells ameliorates the pro-apoptotic profile in MPS IVA fibroblasts.

### 1.5 Decreased Levels of Pro-Inflammatory Markers in MPS IVA Fibroblasts

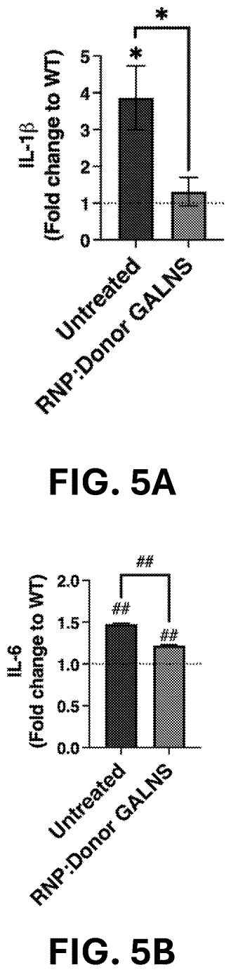

Pro-inflammatory factors such as IL-1β and IL-6 are significantly elevated in MPS IVA and other MPS patients. To assess the effect of CRISPR/nCas9-edited CD34+ cells on the pro-inflammatory profile of co-cultured MPS IVA fibroblasts, ELISA was performed to quantify IL-1β and IL-6 (FIG. 5A, FIG. 5B). MPS IVA fibroblasts were characterized by a significant increase in IL-13 and IL-6 compared to WT fibroblasts. Upon co-culture, MPS IVA fibroblasts showed a decrease in IL-1β (FIG. 5A) and IL-6 levels (FIG. 5B), respectively. Collectively, these findings further support that the CRISPR/nCas9 system mediates cross-correction in MPS IVA fibroblasts, ameliorating pro-inflammatory events in MPS IVA fibroblasts.

### 1.6 Conclusions

Previously, a CRISPR/nCas9-based GT strategy was reported for inserting an expression cassette carrying the GALNS cDNA into the AAVS1 locus in human MPS IVA fibroblasts using non-viral vectors. Although in vitro assessment showed successful HR of the GALNS cDNA expression cassette, leading to the long-term biochemical recovery of the MPS IVA fibroblast phenotype, in vivo assessment using the ROSA26 locus as a safe harbor only partially rescued bone lesions in MPS IVA mice, highlighting the need for a novel strategy. In this regard, previous studies conducted by Gomez-Ospina et al., using the CRISPR/Cas9 system to edit HSPCs at the CCR5 locus, demonstrated that ex-vivo CRISPR/Cas9-based GT could improve bone lesions in MPS I mice upon transplantation. CRISPR/Cas9-edited HSCs can differentiate into diverse blood and immune cell lineages, offering a promising therapeutic approach for systemic pathologies. Recently, ex-vivo LV-based GT was tested in MPS IVA mice, demonstrating partial improvement in bone structure. Nevertheless, random integration into the host genome remains a significant concern that needs to be deeply studied.

In this study, the previously reported CRISPR/nCas9 system was validated in vitro to insert a GALNS expression cassette at the AAVS1 locus in CD34+ cells. Under experimental conditions, long-term supraphysiological GALNS levels were observed in CD34+ cells (FIG. 1C, FIG. 1D) without affecting stemness (FIG. 2). Although the extracellular GALNS levels detected in CD34+ cells in this study were lower than the IDUA levels observed by Gomez-Ospina et al., when using PGK as promoter driving gene expression, lysosomal mass was similarly rescued in both approaches upon co-culture with human MPS fibroblasts (FIG. 3E, FIG. 3F). While extracellular GALNS enzyme activity was evaluated in bulk CD34+ cells in this study, IDUA activity was quantified in sorted CD34+ cells by Gomez-Ospina et al., potentially explaining the differences between both studies. Besides, unlike the IDUA enzyme, which mediates its catabolic activity as a monomer, GALNS requires homodimerization to catabolize substrates effectively, further explaining the differences noticed. Finally, the safe harbor used in this study (AAVS1) and others (e.g., CCR5) could also play critical roles in knock-in approaches. Early reports by others showed that chromatin conformation is important to genome editing efficiency. In this context, such studies reported that while the CCR5 locus and its surrounding regions are characterized by closed chromatin, which hinders Cas9-mediated DNA cutting, the AAVS1 locus remains in open chromatin in stem cells, making it a more suitable target for genome editing than CCR5. GALNS enzyme activity quantification in sorted CD34+ cells will elucidate this premise.

CRISPR/nCas9-edited CD34+ stemness was evaluated, as concerns have been raised about the potential compromise of HSCs' properties after transfection with CRISPR/Cas9 genome strategies. Indeed, functional assessments of CRISPR/Cas9-edited HSCs are essential. The CRISPR/nCas9-edited CD34+ cells maintained their stemness, consistent with previous findings in other CRISPR/Cas9-based genome editing studies. Importantly, these results confirm that the CRISPR/nCas9 strategy did not negatively impact the self-renewal or differentiation capabilities of CD34+ cells. Preserving colony-forming potential and multilineage differentiation suggests that CRISPR/nCas9-edited CD34+ cells retain their sternness, which is crucial for robust engraftment. More significantly, the preservation of sternness in CRISPR/nCas9-edited CD34+ cells is particularly promising, as edited HSCs could provide sustained therapeutic effects post-transplantation by continuously supplying GALNS-expressing blood cells throughout the body.

Ex-vivo GT for LSDs is based on the ability to release therapeutic levels of missing lysosomal enzymes to be uptaken by surrounding cells and supply the deficient enzyme. To evaluate the effect of the GALNS enzyme released by CRISPR/nCas9-edited CD34+ cells, these cells were co-cultured with human MPS IVA fibroblasts for one month and GALNS enzyme activity was assessed along with classical LSD biomarkers. Co-culturing CRISPR/nCas9-edited CD34+ cells with MPS IVA fibroblasts resulted in a recovery of the intracellular GALNS enzyme activity of up to WT levels (FIG. 3C). These results agreed with previous findings on MPS IVA fibroblasts using non-viral vectors.

Consistent with the recovery of GALNS enzyme activity, efficient cross-correction in MPS IVA fibroblasts was observed upon co-culture with CRISPR/nCas9-edited CD34+ cells. Specifically, a reduction in pathological markers such as KS accumulation and lysosomal mass was noted, both of which were normalized to WT levels (FIG. 3D). The reduction in mono-sulfated KS achieved in this study was comparable to previous in vitro studies, where the CRISPR/nCas9 system led to normalization of total GAGs in human MPS IVA fibroblasts. A consistent decrease in lysosomal mass was observed in MPS IVA fibroblasts co-cultured with CRISPR/nCas9-edited CD34+ cells (FIG. 3E, FIG. 3F). These findings agreed with previous findings when using CRISPR/nCas9- and LV-based GT.

Increased pro-oxidant levels and mitochondrial mass are critical biomarkers for evaluating mitochondrial dysfunction in several MPS. Since mitochondria are the primary source of ROS, excessive ROS production is associated with mitochondrial dysfunction. Notably, co-culturing MPS IVA fibroblasts with CRISPR/nCas9-edited CD34+ cells resulted in an improvement in global ROS levels while normalizing mtROS levels to WT levels (FIG. 4A, FIG. 4B). Furthermore, these results demonstrated a reduction in mtROS levels compared to those reported in fibroblasts treated with a non-viral CRISPR/nCas9-strategy GT.

LSDs are characterized by lysosomal dysfunction; therefore, it is reasonable to hypothesize that impaired autophagy/mitophagy could increase mitochondrial mass. In line with this assumption, a significant increase in mitochondrial mass was observed in untreated MPS IVA fibroblasts (FIG. 4C). When MPS IVA fibroblasts were co-cultured with CRISPR/nCas9-edited CD34+ cells, a significant decrease in the mitochondrial mass was detected, suggesting that lysosomal mass recovery may also influence lysosome-related organelles such as mitochondria. Likewise, mitochondrial dysfunction strongly triggers intrinsic apoptosis. Under experimental conditions, untreated MPS IVA fibroblasts exhibited a significant pro-apoptotic profile, which can be reverted upon co-culture with CRISPR/nCas9-edited CD34+ cells (FIG. 4D); further supporting that supplying GALNS enzyme through ex vivo CRISPR/Cas9-based GT could be a promising approach for preserving cell survival. Notably, the increase in apoptosis levels was recently reported by Brokowska et al., supporting these findings for untreated MPS IVA fibroblasts.

Pro-inflammatory events have been described in MPS IVA patients, and they seem not to be ameliorated upon ERT treatment. To assess the pro-inflammatory profile in MPS IVA fibroblasts and evaluate its modulation upon co-culture with CRISPR/nCas9-edited CD34+ cells, the levels of IL-1β and IL-6 were quantified through ELISA (FIG. 5). Both interleukins were significantly elevated in untreated MPS IVA fibroblasts; however, co-culture with CRISPR/nCas9-edited CD34+ cells led to a significant recovery on these parameters, suggesting a positive effect on the pro-inflammatory profile. The impact of the CRISPR/nCas9-based GT in HSCs on the pro-inflammatory profile, mitochondrial mass recovery, and pro-apoptotic still needs to be further addressed in MPS IVA/CD34+ cells and MPS IVA animal models.

This study provides substantial evidence for the therapeutic potential of CRISPR/nCas9-edited HSCs in treating MPS IVA. By editing CD34+ cells to introduce a functional GALNS gene at the AAVS1 locus, sustained GALNS enzyme expression, efficient cross-correction in co-cultured MPS IVA fibroblasts, and recovery of disease-related biomarkers, including GAG accumulation, oxidative stress, apoptosis ratio, and pro-inflammatory profile, were achieved. The CRISPR/Cas9-edited HSCs maintained their self-renewal and differentiation capabilities, which are crucial for ensuring long-term therapeutic benefits following transplantation.

Overall, these results highlight the therapeutic potential of CRISPR/nCas9-edited HSCs as an effective ex vivo gene therapy for MPS IVA. The ability of these CRISPR/nCas9-edited HSCs to differentiate into various blood cell lineages and to secrete the GALNS enzyme throughout the body permanently supposes a therapeutical effect in several affected organs via cross-correction.

### 1.7 Methods

Cell culture and expansion: Human bone marrow CD34+ stem cells were purchased from StemCell Technologies (Vancouver, Canada) and cultured in StemSpan SFEM II medium supplemented with StemSpan CD34+ Expansion Supplement and small molecule UM729. CD34+ were maintained at 37 °C in 5% CO2 for three days to allow sufficient cell expansion before gene editing. Human WT and MPS IVA fibroblasts were obtained from Nemours Children's Health Biobank and cultured in Dulbecco's Modified Eagle Medium (DMEM, Gibco, Waltham, MA) supplemented with fetal bovine serum (FBS) (Gibco) and penicillin-streptomycin (Gibco) at 37 °C and 5% CO2 for two days before co-culture with CD34+. Co-culture was performed in Corning Transwell clear inserts of 0.4 μm pore size polyethylene Terephthalate (PET) membrane and maintained for 30 days at 37 °C, 5% CO2.

Donor template: The donor template was designed according to previous protocols with slight modifications. Two homologous arms to the AAVS1 locus comprising 500 bp each flanked an expression cassette containing the human phosphoglycerate kinase (hPGK) promoter, a Kozak sequence, GALNS cDNA, and the bovine growth hormone (bGH) poly(A) signal sequence (AAV6-Donor). In some experiments, an additional donor carrying the enhanced green fluorescent protein (EGFP) sequence upstream of GALNS cDNA was designed (AAV6-Donor-EGFP). The P2A sequence was added to mediate ribosome skipping between EGFP and GALNS. Donor templates were delivered to cells using Adeno-associated virus type 6 (AAV6). Vectors were synthesized by VectorBuilder Inc. (Chicago, IL).

CRISPR/nCas9 Editing Protocol: CD34+ cells were electroporated with a ribonucleoprotein (RNP) complex comprised of Cas9-D10A nickase protein (nCas9) (Sigma-Aldrich, St. Louis, MO) and PUREedit modified sgRNAs targeting the AAVS1 locus (synthesized by Sigma-Aldrich). The two sgRNA target sequences were previously reported. After three days post-thawing, CD34+ cells were resuspended in the RNP complex at a molar ratio and subsequently electroporated using the 4D-Nucleofector X (Lonza, Walkersville, MD) with the P3 Primary Cell Kit. Following electroporation, the AAV6 donor template was delivered at a multiplicity of infection (MOI). CD34+ cells electroporated with either the RNP complex or donor template (mock) were included as experimental controls.

Donor template integration at the AAVS1 locus: Donor template integration at the AAVS1 locus was evaluated by the EGFP expression in CD34+ cells electroporated with or without the RNP complex, followed by transduction of AAV6-Donor-EGFP. EGFP expression was assessed by flow cytometry after up to 30 days using a NovoCyte 3000 cytometer (Agilent Technologies, Santa Clara, CA). Data were analyzed by FlowJo Software (Becton Dickinson, Franklin Lakes, NJ).

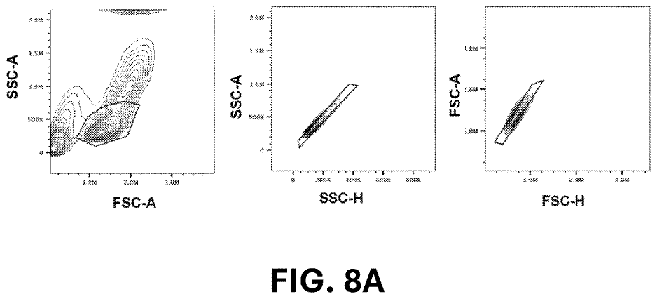

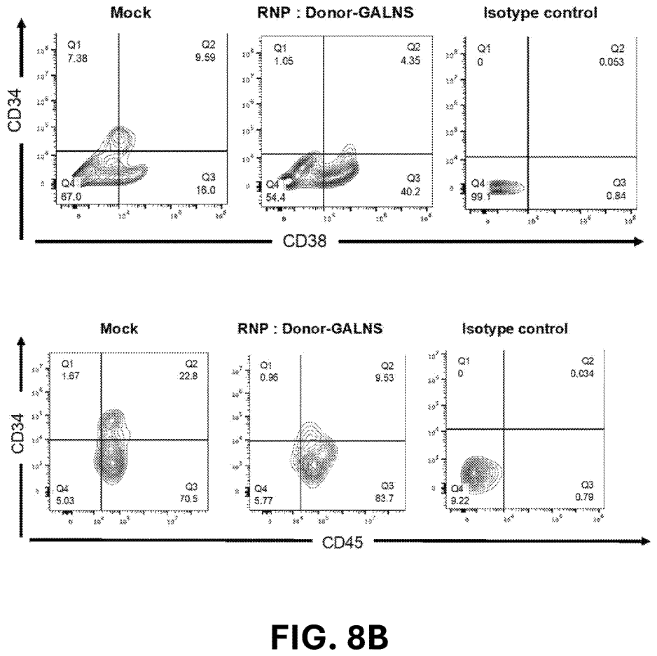

Stemness evaluation, CD Markers: The expression profile of primitive hematopoietic progenitor markers was analyzed in CRISPR/nCas9-edited CD34+ after 15 days post-edition. Cells were stained with anti-human CD34+ APC antibody, anti-human CD38+PE antibody, and anti-human CD45+ FITC antibody. Viable single cells were analyzed using a NovoCyte 3000 cytometer (Agilent Technologies). Gating strategy for the CD34+, CD38+, and CD45+ analysis is shown in FIG. 8. Isotype control antibodies were used as isotype controls.

Stemness evaluation, Colony-forming unit (CFU): CFU experiments were performed to evaluate differentiation potential in CRISPR/nCas-edited CD34+ cells. Upon 14 days post-editing, CRISPR/nCas9-edited CD34+ cells were seeded in MethoCult media on SmartDish culture plate dishes (StemCell Technologies, Vancouver, Canada) at a concentration of cells/mL and followed up to 14 days of incubation. The resulting colonies were classified as colony-forming unit-erythroid (CFU-E), colony-forming unit-granulocyte, erythrocyte, macrophage, megakaryocyte (CFU-GEMM), Burst-forming unit-erythroid (BUF-E), and colony-forming unit-granulocyte, macrophage (CFU-GM). Colony classification was conducted using visual inspection aided by inverted microscopy (AXIO Observer Z1 microscope, Zeiss, Germany). A flow cytometry-based analysis was included to evaluate the differentiation potential of CRISPR/nCas9 edited CD34+ cells using the StemMACS HSC-CFU Assay Kit (Miltenyi Biotec). The total number of colonies and colony type was determined based on the percentages of positive signals for CD15, CD14, and CD235a differentiation markers in viable single cells. The colony type was classified on whether cells exhibited a single positive signal for one marker or positive signals for two or more markers analyzed in a NovoCyte 3000 cytometer (Agilent Technologies). The data obtained was analyzed by FlowJo Software (Becton Dickinson).

Proliferation and cell cycle analysis: The proliferation index and cell cycle of CRISPR/nCas9-edited CD34+ cells were analyzed after 14 days post-editing using 7-Aminoactinomycin D (7-AAD) (Invitrogen, Waltham, MA) and Hoechst 33342 (Invitrogen, Waltham, MA) respectively, following a previously reported protocol with minor modifications. Cells were collected and washed by centrifugation at g for minutes with Hank's Balanced Salt Solution (HBSS, Gibco) supplemented with FBS (Gibco). Cells were then fixed in ethanol for one hour at 4 °C, followed by a wash with HBSS. Subsequently, cells were permeabilized using PTF buffer (Phosphate buffered saline (PBS), Triton X-100, FBS). Cells were stained in 7-AAD- or Hoechst-containing HBSS and incubated at 4 °C in the dark. At least events were collected using excitation/emission: nm (7-AAD) and nm long-pass filter on the NovoCyte 3000 cytometer (Agilent Technologies). The resulting data were analyzed using FlowJo Software (Becton Dickinson).

GALNS activity assays: Specific and volumetric GALNS enzyme activity in MPS IVA fibroblasts and CRISPR/nCas9-edited CD34+ cells was determined as previously described. GALNS activity was measured using 4-MU-Gal-6S. The reaction was incubated at 37 °C for hours, followed by incubation with b-galactosidase for one hour. The enzyme reaction was stopped by glycine-carbonate buffer (pH). The plate was read using excitation/emission in a FLUOstart Omega microplate reader (BMG LabTech, Ortenberg, Germany). Enzyme activity (U) was defined as the amount of enzyme required to hydrolyze substrate per hour, expressed either per milliliter or per milligram of protein, based on a 4-methylumbelliferone standard curve (Sigma-Aldrich). Protein concentration was quantified using a BCA Protein Assay Kit (Thermo Fisher Scientific). Data are presented as fold-change to untreated.

Lysosomal accumulation, Lysosomal mass evaluation: After 30 days of culturing, the lysosomal mass in MPS IVA fibroblasts co-cultured with CRISPR/nCas9-CD34+ cells was determined by staining fibroblasts with LysoTracker Deep Red (Invitrogen), as previously described. Fibroblasts were incubated with LysoTracker in DMEM supplemented with FBS for one hour. Cells were collected by trypsinization and washed with PBS, followed by gentle resuspension in a solution containing Propidium Iodine (PI; Invitrogen) in HBSS plus FBS. Events of viable single fibroblasts were analyzed using a NovoCyte 3000 cytometer (Agilent Technologies) using excitation/emission. Data were analyzed using FlowJo Software (Becton Dickinson). The lysosomal mass was further investigated by epifluorescence microscopy, as previously reported, with minor modifications. MPS IVA fibroblasts were seeded on coverslips pre-coated with Poly-D-Lysine (Gibco) and co-cultured with CRISPR/nCas9-edited CD34+ cells for 30 days. Cells were incubated with LysoTracker Deep Red for one hour, washed with PBS, and fixed in paraformaldehyde in PBS (Invitrogen) for 15 minutes at room temperature. Coverslips were transferred to slides containing Prolong Glass Antifade Mountant with NucBlue Stain (Invitrogen). Images were captured using an inverted fluorescence microscopy (AXIO Observer Z1 microscope) and analyzed using ImageJ.

Lysosomal accumulation, Analysis of mono-sulfated keratan sulfate: Mono-sulfated KS levels in MPS IVA fibroblasts co-cultured with CRISPR/nCas9-edited CD34+ cells were determined by liquid chromatography-tandem mass spectrometry (LC-MS/MS) via enzymatic digestion of KS polymers, as previously described. Protein extracts from fibroblasts, obtained using Pierce Ripa buffer (Thermo Fisher Scientific), were processed with Omega molecular weight cutoff filter plates (Pall Corporation, Port Washington, NY) positioned on a receiving plate. A reaction mixture containing Tris-HCl, chondrosine as the internal standard (IS), heparitinase, and Keratanase II was added to each well. The plate was incubated overnight at 37 °C. After incubation, the samples were centrifuged at 2500×g for 15 minutes. Disaccharides were isolated using a Hypercarb column at 60 °C. A binary gradient elution was used. The mass spectrometer (Agilent Technologies) was operated in negative ion mode with electrospray ionization at a 0.7 mL/min flow rate and a five min. runtime. Disaccharides were detected using m/z ratios, with precursor and product ions for IS and mono-sulfated KS.

Oxidative profile assessment, Reactive oxygen species (ROS): Global oxidative stress was assessed by incubating MPS IVA fibroblasts, co-cultured for one month with CRISPR/nCas9-edited CD34+ cells, in H2DCFDA diluted in DMEM supplemented with FBS. The incubation was performed at 37 °C for 30 minutes. Cells were harvested by trypsinization, washed with PBS, and resuspended in HBSS. Cells were stained with PI and incubated at room temperature for 30 minutes. Viable single cells were analyzed using a NovoCyte 3000 cytometer (Agilent Technologies) with excitation/emission wavelength. Fibroblasts incubated with menadione were used as a positive control.

Oxidative profile assessment, Mitochondrial-derived reactive oxygen species (mtROS): Mitochondrial-dependent oxidative stress in MPS IVA fibroblasts co-cultured with CRISPR/nCas9- and mock-treated CD34+ cells was measured after incubation using MitoSOX mitochondrial superoxide indicator (Invitrogen) as described before. Fibroblasts were stained with MitoSOX diluted in DMEM supplemented with FBS for 15 minutes at 37 °C. Cells were collected by trypsinization, washed with PBS, and resuspended in HBSS. Fibroblasts treated with menadione (Sigma-Aldrich) were included as positive controls. Events of viable single fibroblasts were analyzed using a NovoCyte 3000 cytometer (Agilent Technologies) with 510/580 nm excitation/emission wavelength. Data were analyzed using FlowJo Software (Becton Dickinson).

Mitochondrial mass detection: Mitochondrial mass was evaluated in MPS IVA fibroblasts co-cultured with CRISPR/nCas9-edited CD34+ cells using Nonyl Acridine Orange (NAO, Invitrogen), with 488/530 nm excitation/emission. MPS IVA fibroblasts were incubated with NAO for hour at 37 °C. After washes with HBSS, cells were used for flow cytometry using a NovoCyte 3000 cytometer (Agilent Technologies). Cell viability was evaluated by PI staining. Data were analyzed using FlowJo Software (Becton Dickinson).

Apoptosis ratio: MPS IVA fibroblasts co-cultured with CRISPR/nCas9-edited CD34+ cells were used to quantify apoptosis ratio using Dead Cell Apoptosis Kits with Annexin V assay (Molecular Probes, Eugene, OR) to quantify phosphatidyl serine (PS) externalization. PS was detected using an Alexa Fluor 488-conjugated Annexin V antibody (excitation/emission). Fibroblasts incubated with staurosporine were used as a positive control of apoptosis.

Pro-inflammatory profile evaluation: Enzyme-linked immunosorbent assay (ELISA) assays were conducted to evaluate the levels of IL-1β and IL-6 in MPS IVA fibroblasts co-cultured with CRISPR/nCas9-edited CD34+ cells. An aliquot of media was collected from MPS IVA fibroblasts for one month and stored at −20 °C. Samples were pooled, clarified by centrifugation, and used to determine cytokine levels. Plates were read using a FLUOstart Omega microplate reader (BMG LabTech, Ortenberg, Germany). Absorbance values from MPS IVA fibroblasts were plotted against a standard curve for each cytokine.

Statistical analysis: Statistical tests were processed and analyzed using GraphPad Prism for macOS (GraphPad Software, LLC). Data are reported as mean±standard error (SE). Shapiro-Wilk and Levene's tests were used to evaluate the normal distribution and homoscedasticity. According to normal distribution findings, mean comparisons between groups were made with Student's t-test, Mann-Whitney U test, or one-way analysis of variance (ANOVA).

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the disclosure. It is intended that the following claims define the scope of the embodiments disclosed herein, and that methods and structures within the scope of these claims and their equivalents be covered thereby.

SEQUENCES

| Number | Sequence | Description |
|---|---|---|
| 1 | MAAVVAATRWWQLLLVLSAAGMGASGAPQPPNILLLLMDDMG | GALNS protein |
| | WGDLGVYGEPSRETPNLDRMAAEGLLFPNFYSANPLCSPSRAALLT | |
| | GRLPIRNGFYTTNAHARNAYTPQEIVGGIPDSEQLLPELLKKAGYVS | |
| | KIVGKWHLGHRPQFHPLKHGFDEWFGSPNCHFGPYDNKARPNIP | |
| | VYRDWEMVGRYYEEFPINLKTGEANLTQIYLQEALDFIKRQARHHP | |
| | FFLYWAVDATHAPVYASKPFLGTSQRGRYGDAVREIDDSIGKILELL | |
| | QDLHVADNTFVFFTSDNGAALISAPEQGGSNGPFLCGKQTTFEGG | |
| | MREPALAWWPGHVTAGQVSHQLGSIMDLFTTSLALAGLTPPSDR | |
| | AIDGLNLLPTLLQGRLMDRPIFYYRGDTLMAATLGQHKAHFWTWT | |
| | NSWENFRQGIDFCPGQNVSGVTTHNLEDHTKLPLIFHLGRDPGERF | |
| | PLSFASAEYQEALSRITSVVQQHQEALVPAQPQLNVCNWAVMNW | |
| | APPGCEKLGKCLTPPESIPKKCLWSH | |
| 2 | ATGGCGGCGGTTGTCGCGGCGACGAGGTGGTGGCAGCTGTTGC | GALNS CDS |
| | TGGTGCTCAGCGCCGCGGGGATGGGGGCCTCGGGCGCCCCGCA | |
| | GCCCCCCAACATCCTGCTCCTGCTCATGGACGACATGGGATGGG | |
| | GTGACCTCGGGGTGTATGGAGAGCCCTCCAGAGAGACCCCGAA | |
| | TTTGGACCGGATGGCTGCAGAAGGGCTGCTTTTCCCAAACTTCT | |
| | ATTCTGCCAACCCTCTGTGCTCGCCATCGAGGGCGGCACTGCTCA | |
| | CAGGACGGCTACCCATCCGCAATGGCTTCTACACCACCAACGCC | |
| | CATGCCAGAAACGCCTACACACCGCAGGAGATTGTGGGCGGCA | |
| | TCCCAGACTCGGAGCAGCTCCTGCCGGAGCTTCTGAAGAAGGCC | |
| | GGCTACGTCAGCAAGATTGTCGGCAAGTGGCATCTGGGTCACAG | |
| | GCCCCAGTTCCACCCCCTGAAGCACGGATTTGATGAGTGGTTTG | |
| | GATCCCCCAACTGCCACTTTGGACCTTATGACAACAAGGCCAGG | |
| | CCCAACATCCCTGTGTACAGGGACTGGGAGATGGTTGGCAGATA | |
| | TTATGAAGAATTTCCTATTAATCTGAAGACGGGGGAAGCCAACC | |
| | TCACCCAGATCTACCTGCAGGAAGCCCTGGACTTCATTAAGAGA | |
| | CAGGCACGGCACCACCCCTTTTTCCTCTACTGGGCTGTCGACGCC | |
| | ACGCACGCACCCGTCTATGCCTCCAAACCCTTCTTGGGCACCAGT | |
| | CAGCGAGGGCGGTATGGAGACGCCGTCCGGGAGATTGATGACA | |
| | GCATTGGGAAGATACTGGAGCTCCTCCAAGACCTGCACGTCGCG | |
| | GACAACACCTTCGTCTTCTTCACGTCGGACAACGGCGCTGCCCTC | |
| | ATTTCCGCCCCCGAACAAGGTGGCAGCAACGGCCCCTTTCTGTG | |
| | TGGGAAGCAGACCACGTTTGAAGGAGGGATGAGGGAGCCTGCC | |
| | CTCGCATGGTGGCCAGGGCACGTCACTGCAGGCCAGGTGAGCC | |
| | ACCAGCTGGGCAGCATCATGGACCTCTTCACCACCAGCCTGGCC | |
| | CTTGCGGGCCTGACGCCGCCCAGCGACAGGGCCATTGATGGCCT | |
| | CAACCTCCTCCCCACCCTCCTGCAGGGCCGGCTGATGGACAGGC | |
| | CTATCTTCTATTACCGTGGCGACACGCTGATGGCGGCCACCCTCG | |
| | GGCAGCACAAGGCTCACTTCTGGACCTGGACCAACTCCTGGGAG | |
| | AACTTCAGACAGGGCATTGATTTCTGCCCTGGGCAGAACGTTTC | |
| | AGGGGTCACAACTCACAATCTGGAAGACCACACGAAGCTGCCCC | |
| | TGATCTTCCACCTGGGACGGGACCCAGGGGAGAGGTTCCCCCTC | |
| | AGCTTTGCCAGCGCCGAGTACCAGGAGGCCCTCAGCAGGATCAC | |
| | CTCGGTCGTCCAGCAGCACCAGGAGGCCTTGGTCCCCGCGCAGC | |
| | CCCAGCTCAACGTGTGCAACTGGGCGGTCATGAACTGGGCACCT | |
| | CCGGGCTGTGAAAAGTTAGGGAAGTGTCTGACACCTCCAGAATC | |
| | CATTCCCAAGAAGTGCCTCTGGTCCCACTAG | |

1. A modified CD34 positive (CD34+) cell comprising an exogenous polynucleotide, wherein:
(i) the exogenous polynucleotide comprises a protein coding sequence that encodes a N-acetylgalactosamine-6-sulfatase (GALNS) enzyme; and
(ii) the exogenous polynucleotide is integrated into the genome of the modified CD34+ cell.

2. The modified CD34+ cell of claim 1, wherein the GALNS enzyme comprises an amino acid sequence having at least 90% sequence identity to SEQ ID NO: 1.

3. The modified CD34+ cell of claim 2, wherein the GALNS enzyme hydrolyzes a sulfate group from a 4-methylumbelliferyl-β-D-galactopyranoside-6-sulfate (4MU-Gal-6S) substrate (e.g., in a fluorometric assay).

4. The modified CD34+ cell of claim 1, wherein the exogenous polynucleotide further comprises a promoter sequence, wherein the promoter sequence initiates transcription of the protein coding sequence encoding the GALNS enzyme.

5. The modified CD34+ cell of claim 1, wherein the CD34+ is a CD34+ stem cell.

6. The modified CD34+ cell of claim 5, wherein the CD34+ stem cell is a bone marrow CD34+ stem cell.

7. The modified CD34+ cell of claim 1, wherein the exogenous polynucleotide is integrated into a genomic safe harbor site within the genome.

8. The modified CD34+ cell of claim 7, wherein the genomic safe harbor site is located within an intragenic site and open chromatin.

9. The modified CD34+ cell of claim 1, wherein the modified CD34+ cell secretes the GALNS enzyme.

10. The modified CD34+ cell of claim 1, wherein co-culturing of the modified CD34+ cell with MPS IVA fibroblasts results in an increase in the GALNS activity in the MPS IVA fibroblasts.

11. The modified CD34+ cell of claim 10, wherein the GALNS activity is extracellular GALNS activity, intracellular GALNS activity, or both extracellular GALNS activity and intracellular GALNS activity.

12. The modified CD34+ cell of claim 1, wherein the modified CD34+ cell is capable of forming colony-forming units (CFUs), wherein the CFUs comprise one or more of CFU-GEMM, CFU-GM, BFU-E, CFU-G, and CFU-M.

13. A method of increasing GALNS activity of GALNS-deficient cells, the method comprising:
co-localizing modified CD34+ cells with the GALNS-deficient cells, wherein the modified CD34+ cells comprise an exogenous polynucleotide, wherein:
(i) the exogenous polynucleotide comprises a protein coding sequence that encodes a N-acetylgalactosamine-6-sulfatase (GALNS) enzyme; and
(ii) the exogenous polynucleotide is integrated into the genome of the modified CD34+ cells.

14. The method of claim 13, wherein the method results in increased GALNS activity.

15. The method of claim 14, wherein the GALNS activity is extracellular GALNS activity, intracellular GALNS activity, or both extracellular GALNS activity and intracellular GALNS activity.

16. The method of claim 13, wherein the method results in a reduction in GAG accumulation in the GALNS-deficient cells.

17. The method of claim 16, wherein the GAG comprises keratan sulfate (KS).

18. The method of claim 17, wherein the KS is mono-KS.

19. A method of increasing an amount of GALNS enzyme in an individual having a GALNS deficiency, the method comprising:
administering modified CD34+ cells to the individual, wherein the modified CD34+ cells comprise an exogenous polynucleotide, wherein:
(i) the exogenous polynucleotide comprises a protein coding sequence that encodes a N-acetylgalactosamine-6-sulfatase (GALNS) enzyme; and
(ii) the exogenous polynucleotide is integrated into the genome of the modified CD34+ cells.

20. The method of claim 19, wherein the method results in an increased amount of GALNS in the individual.

21. The method of claim 19, wherein the GALNS deficiency is MPS IVA.


SEQUENCE LISTING (2 sequences)

SEQ ID NO: 1 - protein, 522 aa, Homo sapiens
MAAVVAATRWWQLLLVLSAAGMGASGAPQPPNILLLLMDDMGWGDLGVYGEPSRETPNLDRMAAEGLLFPNFYSANPLCSPSRAALLTGRLPIRNGFYTTNAHARNAYTPQEIVGGIPDSEQLLPELLKKAGYVSKIVGKWHLGHRPQFHPLKHGFDEWFGSPNCHFGPYDNKARPNIPVYRDWEMVGRYYEEFPINLKTGEANLTQIYLQEALDFIKRQARHHPFFLYWAVDATHAPVYASKPFLGTSQRGRYGDAVREIDDSIGKILELLQDLHVADNTFVFFTSDNGAALISAPEQGGSNGPFLCGKQTTFEGGMREPALAWWPGHVTAGQVSHQLGSIMDLFTTSLALAGLTPPSDRAIDGLNLLPTLLQGRLMDRPIFYYRGDTLMAATLGQHKAHFWTWTNSWENFRQGIDFCPGQNVSGVTTHNLEDHTKLPLIFHLGRDPGERFPLSFASAEYQEALSRITSVVQQHQEALVPAQPQLNVCNWAVMNWAPPGCEKLGKCLTPPESIPKKCLWSH

SEQ ID NO: 2 - DNA, 1569 bp, synthetic construct
  source 1..1569: mol_type = other DNA
atggcggcggttgtcgcggcgacgaggtggtggcagctgttgctggtgctcagcgccgcggggatgggggcctcgggcgccccgcagccccccaacatcctgctcctgctcatggacgacatgggatggggtgacctcggggtgtatggagagccctccagagagaccccgaatttggaccggatggctgcagaagggctgcttttcccaaacttctattctgccaaccctctgtgctcgccatcgagggcggcactgctcacaggacggctacccatccgcaatggcttctacaccaccaacgcccatgccagaaacgcctacacaccgcaggagattgtgggcggcatcccagactcggagcagctcctgccggagcttctgaagaaggccggctacgtcagcaagattgtcggcaagtggcatctgggtcacaggccccagttccaccccctgaagcacggatttgatgagtggtttggatcccccaactgccactttggaccttatgacaacaaggccaggcccaacatccctgtgtacagggactgggagatggttggcagatattatgaagaatttcctattaatctgaagacgggggaagccaacctcacccagatctacctgcaggaagccctggacttcattaagagacaggcacggcaccacccctttttcctctactgggctgtcgacgccacgcacgcacccgtctatgcctccaaacccttcttgggcaccagtcagcgagggcggtatggagacgccgtccgggagattgatgacagcattgggaagatactggagctcctccaagacctgcacgtcgcggacaacaccttcgtcttcttcacgtcggacaacggcgctgccctcatttccgcccccgaacaaggtggcagcaacggcccctttctgtgtgggaagcagaccacgtttgaaggagggatgagggagcctgccctcgcatggtggccagggcacgtcactgcaggccaggtgagccaccagctgggcagcatcatggacctcttcaccaccagcctggcccttgcgggcctgacgccgcccagcgacagggccattgatggcctcaacctcctccccaccctcctgcagggccggctgatggacaggcctatcttctattaccgtggcgacacgctgatggcggccaccctcgggcagcacaaggctcacttctggacctggaccaactcctgggagaacttcagacagggcattgatttctgccctgggcagaacgtttcaggggtcacaactcacaatctggaagaccacacgaagctgcccctgatcttccacctgggacgggacccaggggagaggttccccctcagctttgccagcgccgagtaccaggaggccctcagcaggatcacctcggtcgtccagcagcaccaggaggccttggtccccgcgcagccccagctcaacgtgtgcaactgggcggtcatgaactgggcacctccgggctgtgaaaagttagggaagtgtctgacacctccagaatccattcccaagaagtgcctctggtcccactag